SEQUENCE LISTING (10 sequences)

SEQ ID NO: 1 - DNA, 20 bp, synthetic construct
  source 1..20: mol_type = other DNA
ggggtcaacgttgagggggg

SEQ ID NO: 2 - DNA, 22 bp, synthetic construct
  source 1..22: mol_type = other DNA
tgactgtgaacgttcgagatga

SEQ ID NO: 3 - DNA, 20 bp, synthetic construct
  source 1..20: mol_type = other DNA
tccatgacgttcctgatgct

SEQ ID NO: 4 - DNA, 20 bp, synthetic construct
  source 1..20: mol_type = other DNA
tccatgacgttcctgacgtt

SEQ ID NO: 5 - DNA, 22 bp, synthetic construct
  source 1..22: mol_type = other DNA
tcgtcgttttcggcgcgcgccg

SEQ ID NO: 6 - DNA, 24 bp, synthetic construct
  source 1..24: mol_type = other DNA
tcgtcgttttgtcgttttgtcgtt

SEQ ID NO: 7 - DNA, 22 bp, synthetic construct
  source 1..22: mol_type = other DNA
cgtcgttgtcgtttttgtcgtt

SEQ ID NO: 8 - DNA, 21 bp, synthetic construct
  source 1..21: mol_type = other DNA
tcgtcgttttcgcgcgcgccg

SEQ ID NO: 9 - DNA, 26 bp, synthetic construct
  source 1..26: mol_type = other DNA
tcgcgacgttcgcccgacgttcggta

SEQ ID NO: 10 - DNA, 29 bp, synthetic construct
  source 1..29: mol_type = other DNA
tcgcgaacgttcgccgcgttcgaacgcgg

(19) United States
(12) Patent Application Publication
(10) Pub. No.: US 2025/0295763 A1
(43) Pub. Date: Sep. 25, 2025

(54) ADJUVANT COMPOSITION, PHARMACEUTICAL COMPOSITION COMPRISING THE SAME, AND METHOD FOR PREVENTING OR TREATING DISEASE CAUSED BY VIRAL INFECTION, BACTERIAL INFECTION, PROTOZOAL INFECTION OR CANCER

(71) Applicant: ImmunAdd Inc., Taipei (TW)

(72) Inventors: Pi-Hui LIANG, Taipei (TW); You-Yu LIN, Taipei (TW); Chun-Kai CHANG, Taipei (TW)

(21) Appl. No.: 19/067,748

(22) Filed: Feb. 28, 2025

Related U.S. Application Data
(60) Provisional application No. 63/559,603, filed on Feb. 29, 2024.

(51) Int. Cl. A61K 39/39; A61K 9/107; A61K 9/127; A61K 39/00; A61K 39/145; A61K 47/22; A61K 47/26; A61P 31/16; C12N 7/00

(52) U.S. Cl. CPC A61K 39/39; A61K 9/1075; A61K 9/127; A61K 39/145; A61K 47/22; A61K 47/26; A61P 31/16; C12N 7/00; A61K 2039/55555; A61K 2039/55566; A61K 2039/55577; A61K 2039/572; A61K 2039/575; C12N 2760/16134

(57) ABSTRACT

An adjuvant composition is disclosed by the present disclosure. The adjuvant composition comprises: a saponin conjugate represented by formula (I) or a pharmaceutically acceptable salt or solvate thereof; and a TLR agonist, wherein the formula (I) is shown as follows:

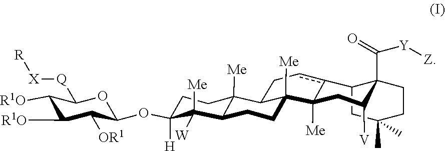

### CROSS REFERENCE TO RELATED APPLICATIONS

The present disclosure claims the benefit of U.S. provisional patent application No. 63/559,603 filed on Feb. 29, 2024, the disclosure of which is incorporated herein by reference.

### INCORPORATION BY REFERENCE OF A SEQUENCE LISTING XML

A Sequence Listing is provided herewith as a Sequence Listing XML, “256002-US_SL_F” created on May 8, 2025 and having a size of 9938 bytes. The contents of the Sequence Listing XML are incorporated by reference herein in their entirety.

### FIELD OF INVENTION

The present disclosure relates to a vaccine adjuvant, and in particular to an adjuvant composition, a pharmaceutical composition comprising the same and a method for preventing or treating disease caused by viral infection, bacterial infection, protozoal infection or cancer.

### BACKGROUND OF INVENTION

Vaccination is one of the most effective and widely employed strategies to prevent and combat infectious and certain non-communicable diseases. Challenges in the field of vaccine development include the need to elicit humoral and cellular immunity, including a pathogen-specific T-cell response, all of which are needed for effective vaccines against various diseases such as HIV, malaria, tuberculosis, and cancers. Adjuvants, which enhance the immune response to antigens, can improve vaccine efficacy and are essential components of many vaccines. However, few adjuvants safe enough for clinical use can induce cellular immunity.

Saponin-based adjuvants have demonstrated exceptional immunostimulatory properties and are known for their ability to induce strong humoral and cellular immune responses. Saponins used in vaccine adjuvants include the Quillaja saponins (Q. saponins), triterpene glycosides isolated from the Chilean soap bark tree Quillaja saponaria Molina. Four major triterpenoid glucosides have been isolated and identified as QS-7, QS-17, QS-18, and QS-21 (Quillaja saponins fraction-7, 17, 18 and 21) from the Quillaja saponaria extract. These saponins all share the same triterpene backbone quillaic acid, flanked by a branched trisaccharide β-D-Gal-(1→2)-[β-D-Xyl-(1→3)]-β-D-GlcA on 3-O position. QS-21 also contains a linear tetrasaccharide moiety β-D-Apif/Xylp-(1→3)-β-D-Xyl-(1→4)-α-L-Rha-(1→2)-β-D-Fuc on 28-O position and a fucose-linked 4-O-acyl stereochemically rich fatty acyl chain 1, where the Apiose and the Xylose substituted moiety is at 65:35 ratio.

QS-7, QS-17, QS-18, and QS-21 are water-soluble saponins, with QS-21 being especially soluble and reported to bind to cholesterol.

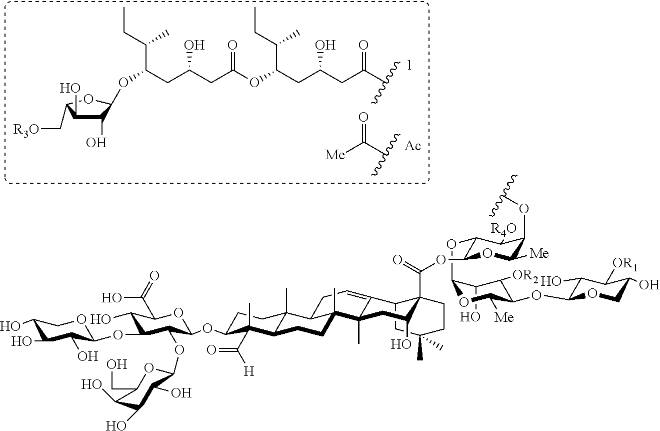

| Compound | Acyl chain | R₁ | R₂ | R₃ | R₄ |
|---|---|---|---|---|---|
| QS-7 | Ac | β-D-Apiƒ | β-D-Glcp | N/A | α-L-Rhap |
| QS-17 | 1 | β-D-Apiƒ | β-D-Glcp | α-L-Rhap | H |
| QS-18 | 1 | β-D-Apiƒ | β-D-Glcp | H | H |
| QS-21ₐₚᵢ | 1 | β-D-Apiƒ | H | H | H |
| QS-21_{xyl} | | β-D-Xylp | | | |

Apiƒ: apiofuranose,
Xylp: xylopyranose,
Glcp: glucopyranose,
Rhap: rhamnopyranose

The favorable adjuvant activities have leaded QS-21 to be used in hundreds of recent and ongoing vaccine clinical trials in many areas, including SARS-CoV-2, malaria, herpes zoster, Alzheimer's disease, HIV-1, respiratory syncytial virus (RSV), melanoma, breast cancer, small cell lung cancer, prostate cancer, etc. In these studies, nature saponins were formulated as liposome or micelle to reduce its toxicity. The most advanced formulation is AS01. GSK's shingles vaccine (Shingrix©) which is adjuvanted by AS01. AS01 is composed of the GSK TLR4 agonist monophosphoryl lipid A (MPL) (purified from Salmonella minnesota) formulated in DOPC/cholesterol liposomes in combination with QS-21. MPL is formulated in a DOPC/cholesterol liposome while QS-21 is prepared in an aqueous formulation and admixed with liposomal MPL, likely resulting in the liposomal incorporation of at least some of the QS-21, as disclosed in U.S. Pat. No. 7,939,084. In 2023, Arexvy (respiratory syncytial virus vaccine, adjuvanted) was approved for the prevention of lower respiratory tract disease (LRTD) caused by RSV in individuals 60 years of age and older. This is the first RSV vaccine for older adults to be approved anywhere in the world and it is also adjuvanted with AS01, albeit lower dose. The clinical report on this vaccine was published in Journal of Infectious Diseases, 2024 Jul. 15; 230(1): e102-e110. While the combination of these two natural products clearly results in a highly immunogenic and efficacious adjuvant, it faces production, scalability, and reactogenicity (specifically injection site pain) challenges. The highest practical tolerable dose in well (non-cancer) adult and child recipients is 25-50 mcg, an immunologically suboptimal dose. As a result, the clinical success of vaccines continues to critically depend on the identification of, and access to, novel, potent adjuvants that are more tolerable.

The AFLQ is also a QS-21-based adjuvant system, which includes monophosphoryl lipid A, as disclosed in U.S. Pat. No. 10,434,167.

Another QS-21-based adjuvant system, AS15, which contains CpG 7909 (a TLR9 agonist), is currently undergoing clinical trials, as published in Journal of Applied Toxicology, 36(2), 238-256.

Another QS-21-based adjuvant system, such as TQL-1055, with the combination of TLR agonists, is disclosed in U.S. Pat. No. 11,324,821B2. This system also published in Human Vaccines & Immunotherapeutics on Jan. 8, 2024; 20(1):2302070.

The widely used of QS adjuvants is limited by their dose-limiting toxicity, inadequate stability, unclear molecular mechanism of action, and the difficulties associated with their procurement. (Expert Rev Vaccines. 2011 April; 10(4):463-470.) Harvest of QS-21 relies on the extract from the tree-bark with low yields, and thus, it faced the problem of unsustainable supply/sourcing. While the isolation of natural products can be scaled up, changes in sourcing of the starting material or location or process in which the starting material is grown or changes to the location of isolating the natural product can result in unacceptable variation in the final product. This results in the inability to produce the adjuvant components at multiple sites or overcome supplier shortages of starting materials, causing both production and scalability challenges. Synthetic saponin (Synsap) of the present disclosure provided a sustainable choice of saponin adjuvant, and offered several advantages, such as structure-well-defined, high purity, and scalable. More importantly, synthetic saponins have a clearer structure-activity relationship compared to natural saponins, which exhibit poly-pharmacological/toxicological effects. A pure synthetic saponin can generate more selective mechanisms of action, making the occurrence of side effects more controllable. When combining immune-enhancing adjuvants with different mechanisms, there is a greater opportunity to design adjuvant systems that can provide various vaccine applications, especially in terms of their efficacy across different ages or antigen activation, leading to more advantageous usage scopes.

### SUMMARY OF INVENTION

The present disclosure provides an adjuvant composition comprising Synsap represented by Formula (I) or a pharmaceutically acceptable salt or solvate thereof, and a TLR agonist, which generate advantageous, and/or synergistic effects. The present disclosure also provides a pharmaceutical composition that includes the elements, along with methods for their creation and use in the treatment and prevention of specific diseases.

In one aspect, the present disclosure provides an adjuvant composition, comprising:
(i) a saponin conjugate represented by formula I or a pharmaceutically acceptable salt or solvate thereof, wherein the formula (I) is shown as follows:

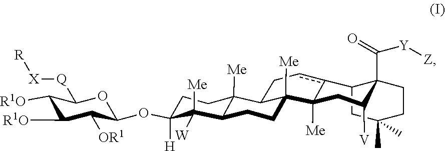

wherein: [symbol] is a single or double bond;
W is Me, —CHO,

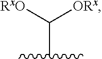

—CH₂OR^{x}, or —C(O)R^{y}, wherein R is independently hydrogen or an oxygen protecting group selected from the group consisting of alkyl ethers, allyl ethers, benzyl ethers, silyl ethers, acetals, ketals, esters, carbamates, and carbonates, and R^{y} is selected from the group consisting of alkyl, allyl, benzyl, silyl, alkoxy and alkylcarboxylate;
V is H or —OH;
Y is CH₂, —O—, —S—, —NR—, or —NH—;
Q is CH₂, C═O, C═N—OH, or C═N—OMe;
X is CH₂, —O—, —NH—, —NH—(C═O)—, —(C═O)—NH—, —S—,
—(C═O)—O— or —O—(C═O)—; R is a moiety selected from the group consisting of aryl, an aryl-aliphatic group, a cyclo-aliphatic group, a heteroaryl-aliphatic group, an alkyloxy-aliphatic group, and an aryloxy-aliphatic group or a moiety selected from the group consisting of an unsubstituted 5-10-membered aryl-aliphatic group, a 5-10-membered aryl-aliphatic group substituted with at least one group selected from the group consisting of an alkyl group, an alkoxy group, an aryloxy group, a halogen, a haloalkyl group, a hydroxyl group, a saturated heterocyclic group having 1-2 heteroatoms independently selected from the group consisting of nitrogen, oxygen, and sulfur, and a combination thereof, and a 4-10-membered heteroaryl-aliphatic group having 1-4 heteroatoms independently selected from the group consisting of nitrogen, oxygen, sulfur, and a combination thereof, or having the following structures:

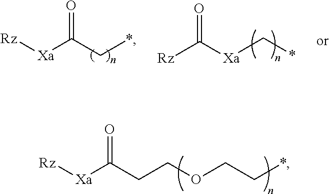

wherein Rz is alkyl, Xa is NH or S, n=1-20;
R¹ is independently hydrogen, an oxygen protecting group selected from the group consisting of alkyl ethers, allyl ethers, benzyl ethers, silyl ethers, acetals, ketals, esters, carbamates, and carbonates, or a carbohydrate having a structure of monosaccharide; and
Z is hydrogen, or a linear or branched oligosaccharide optionally substituted with at least one group selected from the group consisting of amine, amide, acyl, arylalkyl, aryl, heteroaryl, an aliphatic group, a heteroaliphatic group, a cycloaliphatic group and a heterocyclyl group; and
(ii) a TLR agonist.

According to an embodiment of the present disclosure, Z is a linear tetrasaccharide or trisaccharide, and a first sugar residue thereof is attached directly to Y.

According to an embodiment of the present disclosure, Q is C═O and X is —NH—.

According to an embodiment of the present disclosure, W is —CHO and V is —OH.

According to an embodiment of the present disclosure, R¹ is —H.

According to an embodiment of the present disclosure, R is selected from the group consisting of:

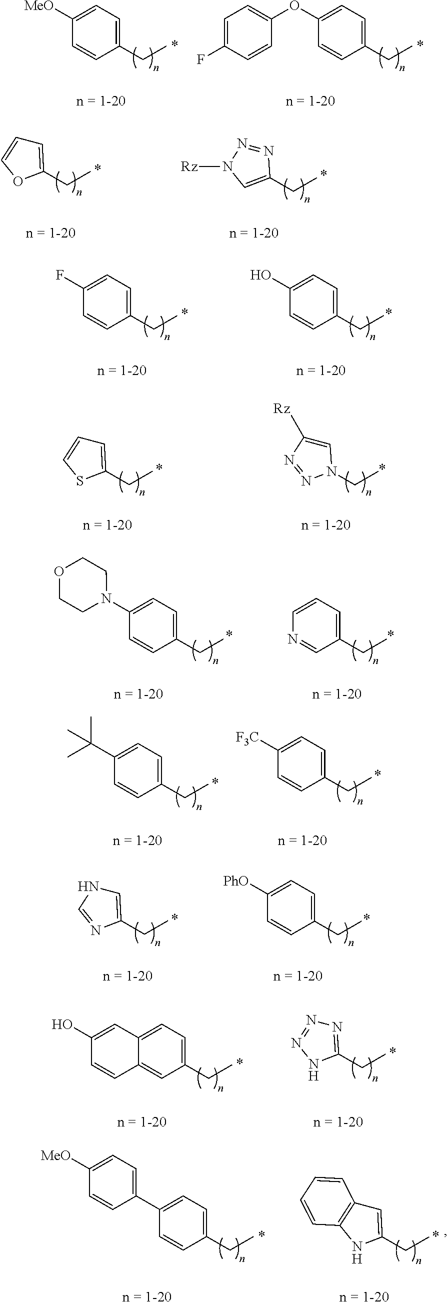

wherein Rz is alkyl.

According to an embodiment of the present disclosure, the saponin conjugate is selected from the group consisting of:
(i) a compound represented by formula I-1:

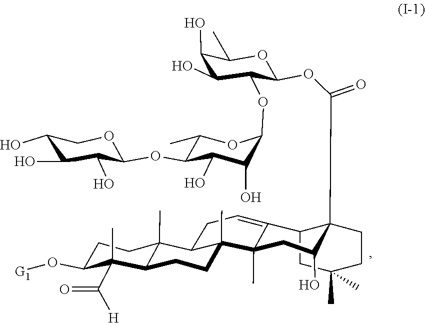

wherein G1 can be 10-((2R,3R,4S,5S,6S)-3,4,5-trihydroxy-6-((8-(4-methoxyphenyl)octyl)carbamoyl)tetrahydro-2H-pyran-2-yl) (Synsap 1), 10-((2R,3R,4S,5S,6S)-6-((8-(4-fluorophenyl)octyl)carbamoyl)-3,4,5-trihydroxytetrahydro-2H-pyran-2-yl) (Synsap 2), 10-((2R,3R,4S,5S,6S)-3,4,5-trihydroxy-6-((8-(4-phenoxyphenyl)octyl)carbamoyl)tetrahydro-2H-pyran-2-yl) (Synsap 3), 10-((2R,3R,4S,5S,6S)-6-((8-(4-(4-fluorophenoxy)phenyl)octyl)carbamoyl)-3,4,5-trihydroxytetrahydro-2H-pyran-2-yl) (Synsap 4), 10-((2R,3R,4S,5S,6S)-3,4,5-trihydroxy-6-((8-(4-morpholinophenyl)octyl)carbamoyl)tetrahydro-2H-pyran-2-yl) (Synsap 51 or 10-((2R,3R,4S,5S,6S)-6-((8-(furan-2-yl)octyl)carbamoyl)-3,4,5-trihydroxytetrahydro-2H-pyran-2-yl) (Synsap 6), but not limited to these compounds;
(ii) compound represented by formula I-2:

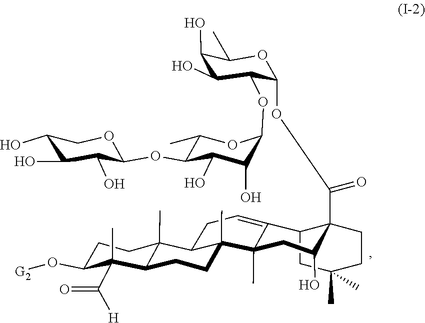

wherein G2 can be 10-((2R,3R,4S,5S,6S)-3,4,5-trihydroxy-6-((8-(4-methoxyphenyl)octyl)carbamoyl)tetrahydro-2H-pyran-2-yl) (Synsap 7), 10-((2R,3R,4S,5S,6S)-6-((8-(4-fluorophenyl)octyl)carbamoyl)-3,4,5-trihydroxytetrahydro-2H-pyran-2-yl) (Synsap 8), 10-((2R,3R,4S,5S,6S)-3,4,5-trihydroxy-6-((8-(4-phenoxyphenyl)octyl)carbamoyl)tetrahydro-2H-pyran-2-yl) (Synsap 9) or 10-((2R,3R,4S,5S,6S)-6-((8-(4-(4-fluorophenoxy)phenyl)octyl)carbamoyl)-3,4,5-trihydroxytetrahydro-2H-pyran-2-yl) (Synsap 10), but not limited to these compounds;
(iii) compound represented by formula I-3:

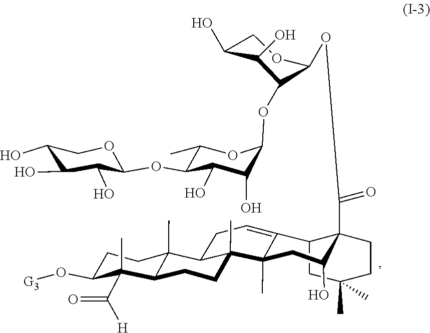

wherein G3 can be 10-((2R,3R,4S,5S,6S)-3,4,5-trihydroxy-6-((8-(4-methoxyphenyl)octyl)carbamoyl)tetrahydro-2H-pyran-2-yl) (Synsap 11), 10-((2R,3R,4S,5S,6S)-6-((8-(4-fluorophenyl)octyl)carbamoyl)-3,4,5-trihydroxytetrahydro-2H-pyran-2-yl) (Synsap 12), 10-((2R,3R,4S,5S,6S)-3,4,5-trihydroxy-6-((8-(4-phenoxyphenyl)octyl)carbamoyl)tetrahydro-2H-pyran-2-yl) (Synsap 13) or 10-((2R,3R,4S,5S,6S)-6-((8-(4-(4-fluorophenoxy)phenyl)octyl)carbamoyl)-3,4,5-trihydroxytetrahydro-2H-pyran-2-yl) (Synsap 14), but not limited to these compounds;
(iv) compound represented by formula I-4:

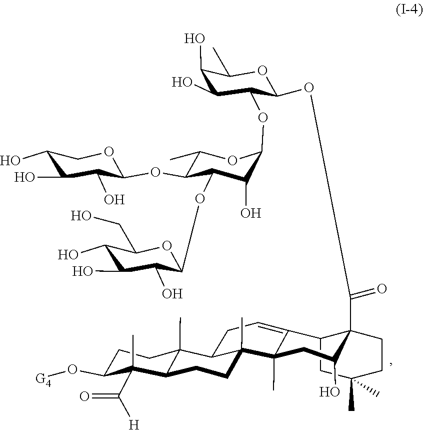

wherein G4 can be 10-((2R,3R,4S,5S,6S)-3,4,5-trihydroxy-6-((8-(4-methoxyphenyl)octyl)carbamoyl)tetrahydro-2H-pyran-2-yl) (Synsap 15), 10-((2R,3R,4S,5S,6S)-6-((8-(4-fluorophenyl)octyl)carbamoyl)-3,4,5-trihydroxytetrahydro-2H-pyran-2-yl) (Synsap 16), 10-((2R,3R,4S,5S,6S)-3,4,5-trihydroxy-6-((8-(4-phenoxyphenyl)octyl)carbamoyl)tetrahydro-2H-pyran-2-yl) (Synsap 17) or 10-((2R,3R,4S,5S,6S)-6-((8-(4-(4-fluorophenoxy)phenyl)octyl)carbamoyl)-3,4,5-trihydroxytetrahydro-2H-pyran-2-yl) (Synsap 18), but not limited to these compounds; and
(v) compound represented by formula I-5:

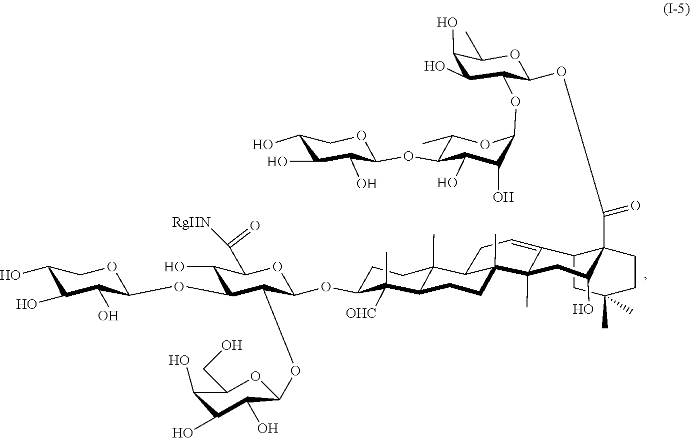

wherein Rg can be 4-methoxyphenyloctyl (Synsap 19), 4-fluorophenyloctyl (Synsap 20), 4-phenoxyphenyloctyl (Synsap 21) or (4-fluorophenoxy)phenyl)octyl (Synsap 22), but not limited to these compounds.

According to an embodiment of the present disclosure, the TLR agonist is selected from the group consisting of a TLR2 agonist, a TLR3 agonist, a TLR4 agonist, a TLR7 agonist, a TLR8 agonist and a TLR9 agonist.

According to an embodiment of the present disclosure, the TLR agonist is a TLR4 agonist selected from the group consisting of PHAD, MPLA, 3D-PHAD, 3D-6A-PHAD, EcML, CRX-527, GSK1795091, E6020, GLA, SLA and a combination thereof.

According to an embodiment of the present disclosure, the adjuvant composition comprises liposome-forming compound or emulsion containing compound.

According to an embodiment of the present disclosure, the liposome-forming compound is selected from the group of phospholipids, such as 1,2-dioleoyl-sn-glycero-3-phosphocholine (DOPC), 1,2-dimyristoyl-sn-glycero-3-phosphocholine (DMPC), dioleoyl phosphatidylglycerol (DOPG), dimyristoyl phosphatidylglycerol (DMPG), 1,2-dioleoyloxy-3-(trimethylammonium)propane (DOTAP), cholesterol or a combination thereof.

According to an embodiment of the present disclosure, the emulsion containing compound is selected from the group consisting of polysorbate, α-tocopherol, span 85, glycerol, poloxamer 188 and carboxymethyl cellulose.

According to an embodiment of the present disclosure, the adjuvant composition is in aqueous solution, in a form of an oil in water emulsion or is encapsulated in a liposome.

According to an embodiment of the present disclosure, a particle size of the adjuvant composition is smaller than 250 nm.

According to an embodiment of the present disclosure, the adjuvant composition further comprises squalene.

In another aspect, the present disclosure further provides a vaccine composition comprising an antigen and an adjuvant composition as mentioned above, and optionally, a pharmaceutically acceptable excipient.

According to an embodiment of the present disclosure, the antigen is selected from one or more of the groups consisting of bacterial, viral, protozoal, and tumor-related antigens.

In another aspect, the present disclosure further provides a method for treating or preventing a disease caused by viral infection, bacterial infection, protozoal infections or cancers, comprising a step of administering the vaccine composition as mentioned above to a subject in need.

In another aspect, the present disclosure provides an adjuvant composition that induces the immune response toward humoral immunity and cellular immunity.

In another aspect, the present disclosure provides a pharmaceutical composition comprising an antigen, an adjuvant composition of the present disclosure, and optionally a pharmaceutically acceptable excipient. In certain embodiments, the pharmaceutical composition is a vaccine comprising an antigen and an adjuvant composition of the present disclosure.

In another aspect, the present disclosure provides formulations incorporating saponin derivatives, their salt forms or their solvate forms. In specific embodiments, the solvent for the formulation may encompass water, alcohols (including, but not limited to, methanol, ethanol, butanol, etc.), polyols (including, but not limited to, glycerol, propylene glycol, polyethylene glycol, etc.), N-methyl-2-pyrrolidone and appropriate mixtures thereof, as well as vegetable oils like olive oil and injectable organic esters such as ethyl oleate.

In another aspect, the present disclosure provides formulations of compositions according to the present disclosure in an adjuvant system. In some embodiments, the adjuvant system utilizes a carrier. In some embodiments, the carrier is a particulate carrier such as metallic salt particles, emulsions (e.g., oil-in-water emulsions), polymers, liposomes, or immune stimulating complexes (ISCOMs).

In another aspect, the present disclosure provides a method of potentiating an immune response to an antigen, comprising administering to a subject a provided vaccine in an effective amount to potentiate the immune response of said subject to said antigen.

In another embodiment, the present disclosure provides a method of stimulating or enhancing cytokine production in a subject, the method includes, inter alia, administering to the subject any one of the pharmaceutical compositions or combinations according to the present disclosure, whereby immune cell secreted cytokines.

In another aspect, the present disclosure provides methods of vaccinating a subject, comprising administering a provided vaccine to said subject. In some embodiments, the subject is human. In some embodiments, the vaccine is administered orally. In other embodiments, the vaccine is administered intramuscularly. In other embodiments, the vaccine is administered subcutaneously. In certain embodiments, the antigen to which the subject is vaccinated may be cancer, bacterial, viral, protozoal, or self-antigen.

In another aspect, the present disclosure provides kits comprising pharmaceutical compositions or combinations of inventive compounds. In some embodiments, the kits comprise prescribing information. In some embodiments, such kits include the combination of inventive adjuvant compounds and other immunotherapeutic agents (e.g. vaccine, antibody). The agents may be packaged separately or together. The kit optionally includes instructions for prescribing the medication. In certain embodiments, the kit includes multiple doses of each agent. The kit may include sufficient quantities of each component to treat a subject for a week, two weeks, three weeks, four weeks, or multiple months. In certain embodiments, the kit includes one cycle of immunotherapy. In certain embodiments, the kit includes a sufficient quantity of a pharmaceutical composition to immunize a subject against an antigen long term.

### DESCRIPTION OF DRAWINGS

To better illustrate the above contents of the present disclosure, the following is a detailed description of the preferred embodiments with reference to the accompanying drawings:

FIGS. 1A-1C show the anti-H3N2 antibody titer measured on Day 28 and expressed as the dilution of serum exhibiting half-maximal binding (optical density: 50%) based on its absorbance at 450 nm, and represent the results from Example 1.

FIG. 2 shows analysis of H3N2-specific CD4⁺ T cell subsets for each group as discussed in Example 1.

FIG. 3 shows the level of IFN-γ for each group as discussed in Example 1 measured via ELISA assay after H3N2 stimulation.

FIG. 4 shows the level of IL-4 for each group as discussed in Example 1 measured via ELISA assay after H3N2 stimulation.

FIGS. 5A-5C show the anti-H3N2 antibody titer measured on Day 28 and expressed as the dilution of serum exhibiting half-maximal binding (optical density: 50%) based on its absorbance at 450 nm, and represent the results from Example 1.

FIG. 6 shows analysis of H3N2-specific CD4⁺ T cell subsets for each group as discussed in Example 1.

FIGS. 7A-7C show the anti-H3N2 antibody titer measured on Day 28 and expressed as the dilution of serum exhibiting half-maximal binding (optical density: 50%) based on its absorbance at 450 nm, and represent the results from Example 2.

FIGS. 8A-8C show the anti-H3N2 antibody titer measured on Day 14 and Day 28 and expressed as the dilution of serum exhibiting half-maximal binding (optical density: 50%) based on its absorbance at 450 nm, and represent the results from Example 3.

FIG. 9 shows the level of IFNγ for each group as discussed in Example 3 measured via ELISA assay after H3N2 stimulation.

FIG. 10 shows the level of IL-4 for each group as discussed in Example 3 measured via ELISA assay after H3N2 stimulation.

FIG. 11 shows the level of IL-17A for each group as discussed in Example 3 measured via ELISA assay after H3N2 stimulation.

FIGS. 12A-12C show the anti-H3N2 antibody titer measured on Day 28 and expressed as the dilution of serum exhibiting half-maximal binding (optical density: 50%) based on its absorbance at 450 nm, and represent the results from Example 4.

FIG. 13 shows analysis of H3N2-specific CD4+ T cell subsets for each group as discussed in Example 4.

FIG. 14 shows the level of IFN-γ for each group as discussed in Example 4 measured via ELISA assay after H3N2 stimulation.

FIG. 15 shows the level of IL-4 for each group as discussed in Example 4 measured via ELISA assay after H3N2 stimulation.

FIGS. 16A-16C show the anti-H3N2 antibody titer measured on Day 28 and expressed as the dilution of serum exhibiting half-maximal binding (optical density: 50%) based on its absorbance at 450 nm, and represent the results from Example 5.

FIG. 17 shows analysis of H3N2-specific CD4⁺ T cell subsets for each group as discussed in Example 5.

FIG. 18 shows the level of IFN-γ for each group as discussed in Example 5 measured via ELISA assay after H3N2 stimulation.

FIG. 19 shows the level of IL-4 for each group as discussed in Example 5 measured via ELISA assay after H3N2 stimulation.

### DETAILED DESCRIPTION OF PREFERRED EMBODIMENTS

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will be controlled. In addition, the materials, methods, and examples are illustrative only and are not intended to be necessarily limiting.

According to an embodiment of the present disclosure, an adjuvant composition comprises:
(i) a saponin conjugate represented by formula (I) or a pharmaceutically acceptable salt or solvate thereof, wherein the formula (I) is shown as follows:

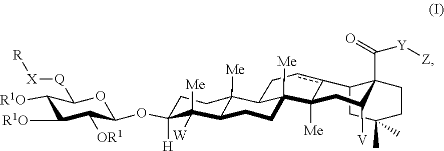

wherein: [symbol] is a single or double bond;
W is Me, —CHO,

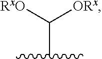

—CH2OR^{x}, or C(O)R^{y}, wherein R is independently hydrogen or an oxygen protecting group selected from the group consisting of alkyl ethers, allyl ethers, benzyl ethers, silyl ethers, acetals, ketals, esters, carbamates, and carbonates, and R^{y} is selected from the group consisting of alkyl, allyl, benzyl, silyl, alkoxy and alkylcarboxylate;
V is H or —OH;
Y is CH₂, —O—, —S—, —NR—, or —NH—;
Q is CH₂, C═O, C═N—OH, or C═N—OMe;
X is CH₂, —O—, —NH—, —NH—(C═O)—, —(C═O)—NH—, —S—,
—(C═O)—O— or —O—(C═O)—;
R is a moiety selected from the group consisting of aryl, an aryl-aliphatic group, a cyclo-aliphatic group, a heteroaryl-aliphatic group, an alkyloxy-aliphatic group, and an aryloxy-aliphatic group or a moiety selected from the group consisting of an unsubstituted 5-10-membered aryl-aliphatic group, a 5-10-membered aryl-aliphatic group substituted with at least one group selected from the group consisting of an alkyl group, an alkoxy group, an aryloxy group, a halogen, a haloalkyl group, a hydroxyl group, a saturated heterocyclic group having 1-2 heteroatoms independently selected from the group consisting of nitrogen, oxygen, and sulfur, and a combination thereof, and a 4-10-membered heteroaryl-aliphatic group having 1-4 heteroatoms independently selected from the group consisting of nitrogen, oxygen, sulfur, and a combination thereof, or having the following structures:

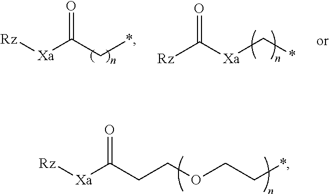

wherein Rz is alkyl, Xa is NH or S, n=1-20;
R¹ is independently hydrogen, an oxygen protecting group selected from the group consisting of alkyl ethers, allyl ethers, benzyl ethers, silyl ethers, acetals, ketals, esters, carbamates, and carbonates, or a carbohydrate having a structure of monosaccharide; and
Z is hydrogen, or a linear or branched oligosaccharide optionally substituted with at least one group selected from the group consisting of amine, amide, acyl, arylalkyl, aryl, heteroaryl, an aliphatic group, a heteroaliphatic group, a cycloaliphatic group and a heterocyclyl group; and
(ii) a TLR agonist.

In some embodiments, Z is a linear tetrasaccharide or trisaccharide, and a first sugar residue thereof is attached directly to Y

In some embodiments, Q is C═O and X is —NH—.

In some embodiments, W is —CHO and V is —OH.

In some embodiments, R¹ is —H.

In some embodiments, R is selected from the group consisting of:

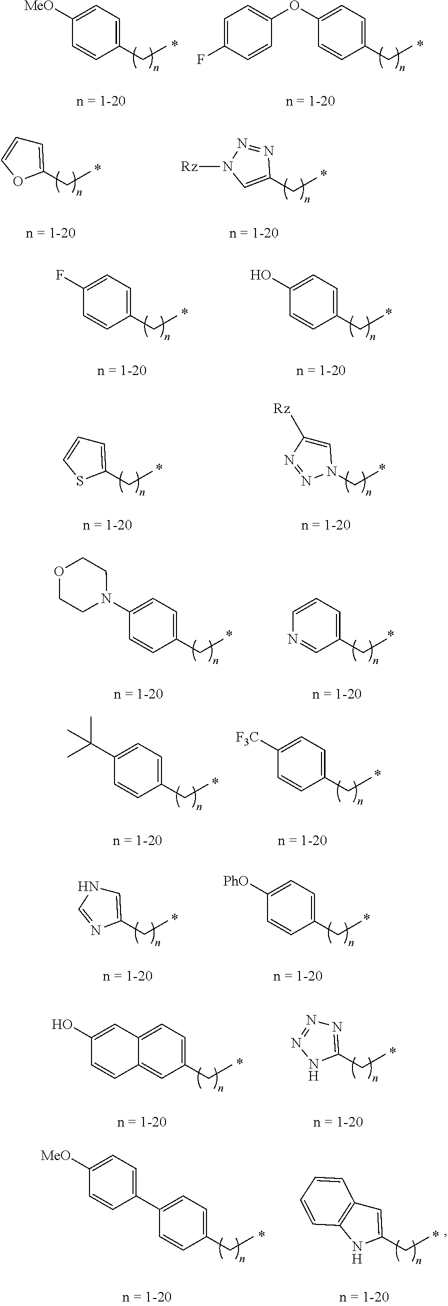

wherein Rz is alkyl.

In some embodiments, the saponin conjugate is selected from the group consisting of:
(i) a compound represented by formula I-1:

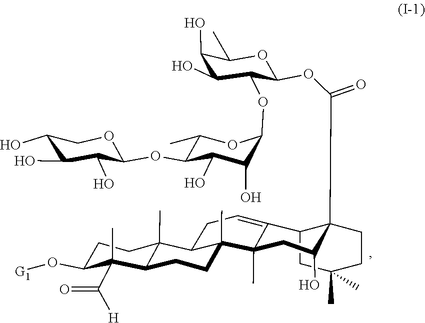

wherein G₁ is 10-((2R,3R,4S,5S,6S)-3,4,5-trihydroxy-6-((8-(4-methoxyphenyl)octyl)carbamoyl)tetrahydro-2H-pyran-2-yl) (Synsap 1), 10-((2R,3R,4S,5S,6S)-6-((8-(4-fluorophenyl)octyl)carbamoyl)-3,4,5-trihydroxytetrahydro-2H-pyran-2-yl) (Synsap 2), 10-((2R,3R,4S,5S,6S)-3,4,5-trihydroxy-6-((8-(4-phenoxyphenyl)octyl)carbamoyl)tetrahydro-2H-pyran-2-yl) (Synsap 3), 10-((2R,3R,4S,5S,6S)-6-((8-(4-(4-fluorophenoxy)phenyl)octyl)carbamoyl)-3,4,5-trihydroxytetrahydro-2H-pyran-2-yl) (Synsap 4), 10-((2R,3R,4S,5S,6S)-3,4,5-trihydroxy-6-((8-(4-morpholinophenyl)octyl)carbamoyl)tetrahydro-2H-pyran-2-yl) (Synsap 5) or 10-((2R,3R,4S,5S,6S)-6-((8-(furan-2-yl)octyl)carbamoyl)-3,4,5-trihydroxytetrahydro-2H-pyran-2-yl) (Synsap 6), but not limited to these compounds;
(ii) a compound represented by formula I-2:

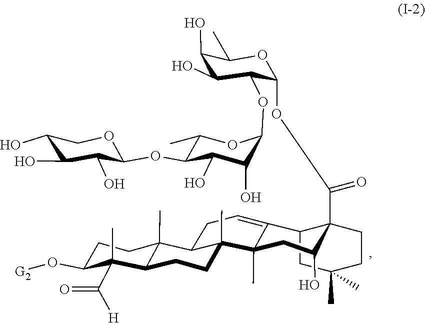

wherein G₂ is 10-((2R,3R,4S,5S,6S)-3,4,5-trihydroxy-6-((8-(4-methoxyphenyl)octyl)carbamoyl)tetrahydro-2H-pyran-2-yl) (Synsap 7), 10-((2R,3R,4S,5S,6S)-6-((8-(4-fluorophenyl)octyl)carbamoyl)-3,4,5-trihydroxytetrahydro-2H-pyran-2-yl) (Synsap 8), 10-((2R,3R,4S,5S,6S)-3,4,5-trihydroxy-6-((8-(4-phenoxyphenyl)octyl)carbamoyl)tetrahydro-2H-pyran-2-yl) (Synsap 9) or 10-((2R,3R,4S,5S,6S)-6-((8-(4-(4-fluorophenoxy)phenyl)octyl)carbamoyl)-3,4,5-trihydroxytetrahydro-2H-pyran-2-yl) (Synsap 10);
(iii) a compound represented by formula I-3:

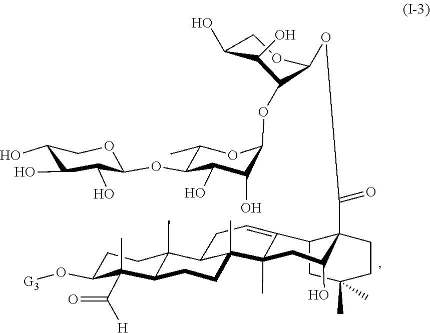

wherein G₃ is 10-((2R,3R,4S,5S,6S)-3,4,5-trihydroxy-6-((8-(4-methoxyphenyl)octyl)carbamoyl)tetrahydro-2H-pyran-2-yl) (Synsap 11), 10-((2R,3R,4S,5S,6S)-6-((8-(4-fluorophenyl)octyl)carbamoyl)-3,4,5-trihydroxytetrahydro-2H-pyran-2-yl) (Synsap 12), 10-((2R,3R,4S,5S,6S)-3,4,5-trihydroxy-6-((8-(4-phenoxyphenyl)octyl)carbamoyl)tetrahydro-2H-pyran-2-yl) (Synsap 13) or 10-((2R,3R,4S,5S,6S)-6-((8-(4-(4-fluorophenoxy)phenyl)octyl)carbamoyl)-3,4,5-trihydroxytetrahydro-2H-pyran-2-yl) (Synsap 14);
(iv) a compound represented by formula I-4:

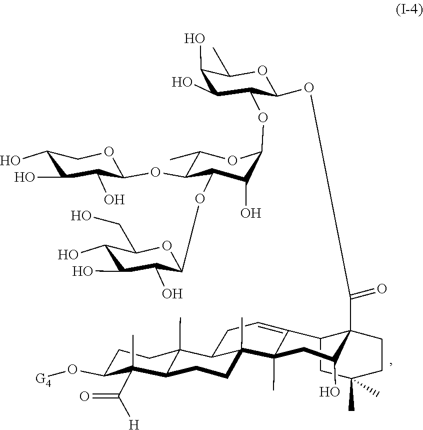

wherein G₄ is 10-((2R,3R,4S,5S,6S)-3,4,5-trihydroxy-6-((8-(4-methoxyphenyl)octyl)carbamoyl)tetrahydro-2H-pyran-2-yl) (Synsap 15), 10-((2R,3R,4S,5S,6S)-6-((8-(4-fluorophenyl)octyl)carbamoyl)-3,4,5-trihydroxytetrahydro-2H-pyran-2-yl) (Synsap 16), 10-((2R,3R,4S,5S,6S)-3,4,5-trihydroxy-6-((8-(4-phenoxyphenyl)octyl)carbamoyl)tetrahydro-2H-pyran-2-yl) (Synsap 17) or 10-((2R,3R,4S,5S,6S)-6-((8-(4-(4-fluorophenoxy)phenyl)octyl)carbamoyl)-3,4,5-trihydroxytetrahydro-2H-pyran-2-yl) (Synsap 18); and
(v) a compound represented by formula I-5:

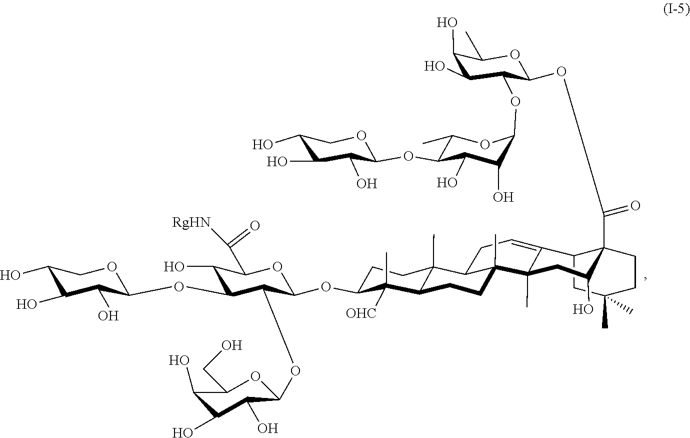

wherein Rg is 4-methoxyphenyloctyl (Synsap 19), 4-fluorophenyloctyl (Synsap 20), 4-phenoxyphenyloctyl ((Synsap 21) or (4-fluorophenoxy)phenyl)octyl (Synsap 22).

In some embodiments, the TLR agonist is selected from the group consisting of a TLR2 agonist, a TLR3 agonist, a TLR4 agonist, a TLR7 agonist, a TLR8 agonist, and a TLR9 agonist.

In some embodiments, the TLR agonist is a TLR4 agonist selected from the group consisting of PHAD, MPLA, 3D-PHAD, 3D-(6A)-PHAD, EcML, CRX-527, GSK1795091, E6020, GLA, SLA and a combination thereof.

In some embodiments, the adjuvant composition comprises liposome-forming compound or emulsion containing compound.

In some embodiments, the emulsion containing compound is selected from the group consisting of polysorbate, α-tocopherol, span 85, glycerol, poloxamer 188 and carboxymethyl cellulose.

In some embodiments, the liposome-forming compound is selected from the group consisting of phospholipids, such as 1,2-dioleoyl-sn-glycero-3-phosphocholine (DOPC), 1,2-dimyristoyl-sn-glycero-3-phosphocholine (DMPC), dioleoyl phosphatidylglycerol (DOPG), dimyristoyl phosphatidylglycerol (DMPG), 1,2-dioleoyloxy-3-(trimethylammonium)propane (DOTAP), cholesterol or a combination thereof.

In some embodiments, the adjuvant composition is in aqueous solution, in a form of an oil in water emulsion or is encapsulated in a liposome.

In some embodiments, a particle size of the adjuvant composition is smaller than 250 nm. Preferably, a particle size of the adjuvant composition ranges from 50 to 200 nm.

In some embodiments, the adjuvant composition further comprises squalene.

According to another embodiment of the present disclosure, a pharmaceutical composition comprising an antigen and an adjuvant composition as mentioned above.

In some embodiments, the pharmaceutical composition is a vaccine.

In some embodiments, the antigen is selected from one or more of the groups consisting of bacterial, viral, protozoal, and tumor-related antigens.

According to a further embodiment of the present disclosure, a method for preventing or treating a disease caused by viral infection, bacterial infection, protozoal infections or cancers, comprising a step of administering the pharmaceutical composition as mentioned above to a subject in need.

### Toll-Like Receptor (TLR) Agonists as Adjuvants

Toll-like receptors (TLRs) are transmembrane pattern recognition receptors that detect invading pathogens and initiate the innate and adaptive immune response. Different TLRs are expressed on distinct subsets of immune and non-immune cell types, including macrophages, dendritic cells, B cells, T cells, fibroblasts, and epithelial cells.

The human TLR2 is expressed as a heterodimer on the surface of plasma membranes, notably on diverse immune cell types, encompassing neutrophils, macrophages, monocytes, basophils, T cells, B cells, natural killer (NK) cells, and immature dendritic cells (DCs). TLR2 is characterized by two distinct configurations: TLR2/6 and TLR2/1. The engagement of TLR2/TLR1 agonists, including Pam3CysSerLys4 (Pam3CSK4) and its derivatives, plays a critical role in enhancing B-cell responses and inducing T-cell stimulation. This underscores their efficacy across a spectrum of experimental vaccine models.

In some embodiments, exemplary TLR2 agonists are described below:

| TLR2 | |
|---|---|
| agonists | Structure |
| Pam3Cys SerLys4 | 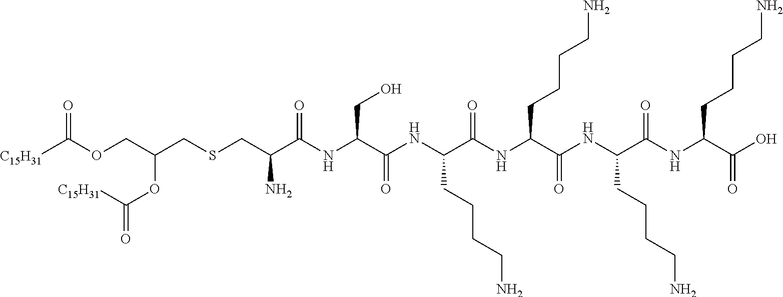 |
| CU-T12-9 | 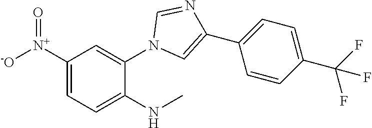 |
| FSL-1 | 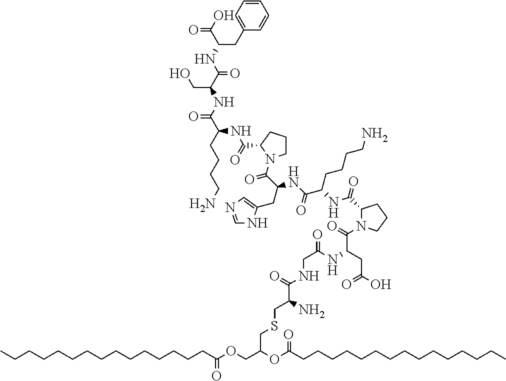 [SMILES: CCCCCCCCCCCCCCCC(=O)OCC(CSC[C@H](N)C(=O)NCC(=O)N[C@@H](CC(=O)O)C(=O)N1CCC[C@H]1C(=O)N[C@@H](CCCCN)C(=O)N[C@@H](Cc1cnc[nH]1)C(=O)N1CCC[C@H]1C(=O)N[C@@H](CCCCN)C(=O)N[C@@H](CO)C(=O)N[C@@H](Cc1ccccc1)C(=O)O)OC(=O)CCCCCCCCCCCCCCC] |

TLR3 is located within endosomes where it recognizes dsRNA produced during viral replication within an infected cell. TLR3 is unique among known TLRs because it signals to activate NF-κB-regulated gene expression. This signaling cascade leads to a T-helper 1 (Th1) polarizing immune response which provided protection against viral infection, making TLR3 agonists attractive adjuvant candidates in vaccines designed to combat intracellular pathogens. The dsRNA mimic poly(I:C) induces NF-κB activation and results in IFN-β induction through TLR3 ligation.

In some embodiments, exemplary TLR3 agonists are described below:

| TLR3 | |
|---|---|
| agonists | Structure |
| Poly (I:C) | 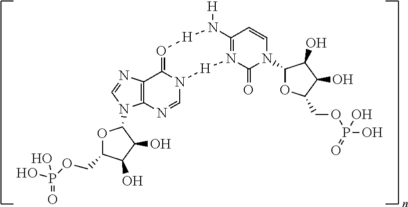 |
| Nexa | novel dsRNA-based TLR3 agonist (275 kDa) |
| Vant | |

TLR4 agonists are recognized for their innate immune-stimulating capabilities, triggering a cascade of signaling events that culminate in the activation of the adaptive immune system. Common TLR4 agonists include biological source monophosphoryl lipid A (MPLA) derivatives such as EcML, and synthetic MPLA-like structures such as PHAD, and CRX-527. Combination vaccine adjuvants comprising TLR4 agonists with saponins have the potential to elicit more robust, targeted and balanced immune responses.

In some embodiments, exemplary TLR4 agonists are described below:

| TLR4 agonists | Structure |
|---|---|
| MPLA | 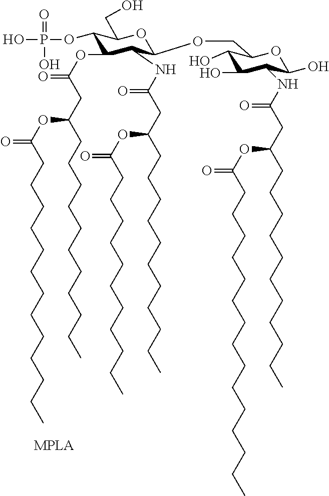 |
| PHAD | 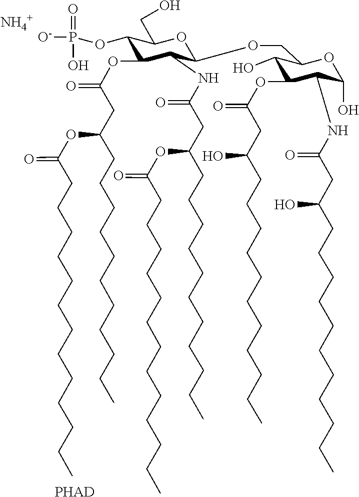 |
| 3D-PHAD | 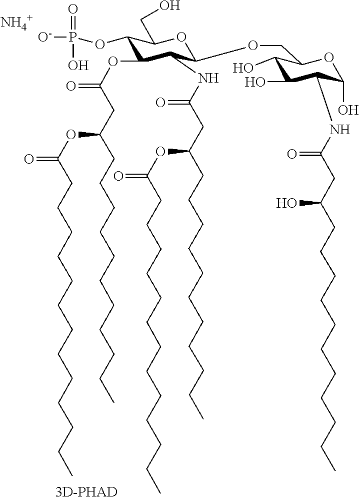 |
| 3D-6A-PHAD | 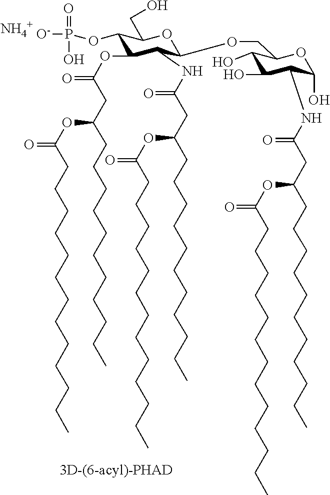 |
| EcML | 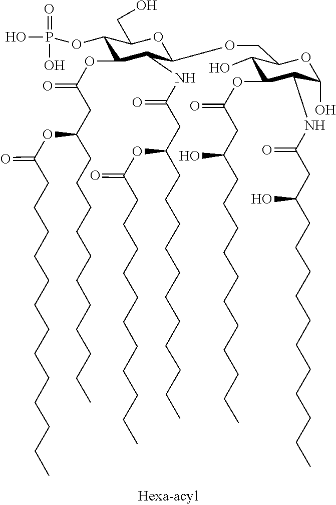 |
| | 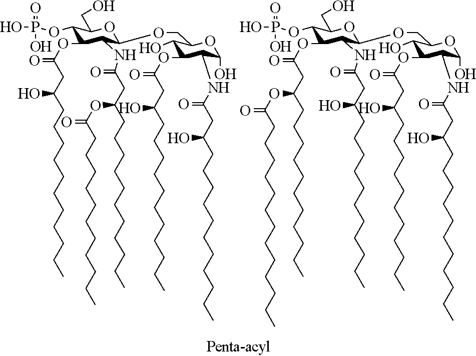 |
| CRX-527 | 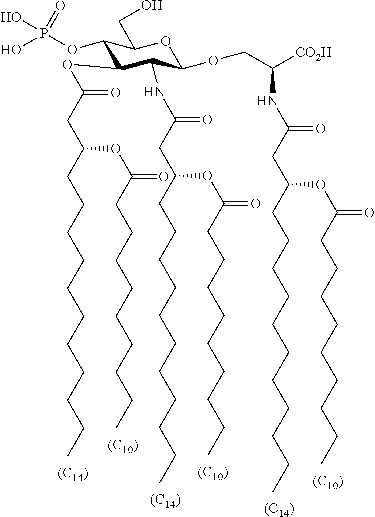 |
| GSK1795091 (CRX-601) | 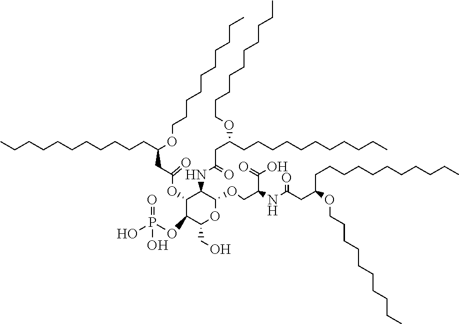 [SMILES: CCCCCCCCCCC[C@H](CC(=O)N[C@H]1[C@H](OC[C@H](NC(=O)C[C@@H](CCCCCCCCCCC)OCCCCCCCCCC)C(=O)O)O[C@H](CO)[C@@H](OP(=O)(O)O)[C@@H]1OC(=O)C[C@@H](CCCCCCCCCCC)OCCCCCCCCCC)OCCCCCCCCCC] |
| E6020 | 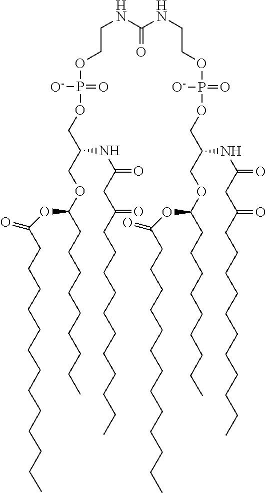 [SMILES: CCCCCCCCCCCCCC(=O)O[C@H](CCCCCCCCC)OC[C@H](COP(=O)([O-])OCCNC(=O)NCCOP(=O)([O-])OC[C@@H](CO[C@@H](CCCCCCCCC)OC(=O)CCCCCCCCCCCCC)NC(=O)CC(=O)CCCCCCCCCCC)NC(=O)CC(=O)CCCCCCCCCCC] |
| GLA | 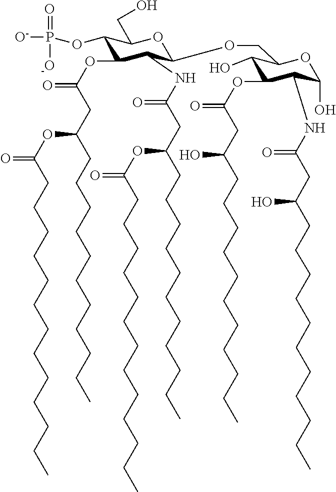 |
| SLA | 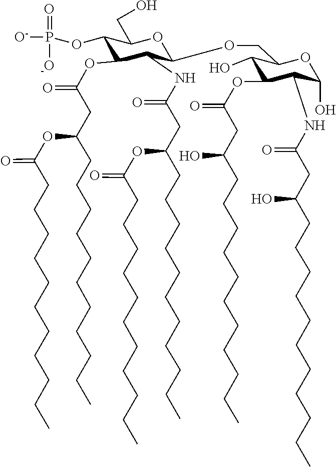 |
| 2002 | 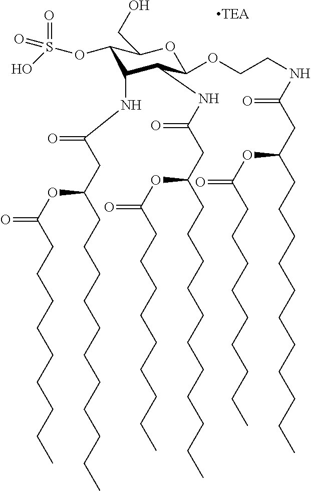 |

TLR7/8 agonists, such as imiquimod and resiquimod have been approved by the FDA for use as stand-alone entities. Imiquimod is approved for topical administration for the treatment of basal cell carcinoma in immunocompetent adults and demonstrates efficacy for treatment of non-genital warts caused by HPV, molluscum contagiosum, squamous cell carcinoma, and lentigo maligna. As predicted for a TLR7/8 agonist, imiquimod stimulates the local induction of IFN-α, TNF, IL-6, and IL-12 at the administration site and induces a cytotoxic T-cell response.

In some embodiments, exemplary TLR7/8 agonists are described below:

| TLR7/8 agonists | Structure |
|---|---|
| Resiquimod (R848) | 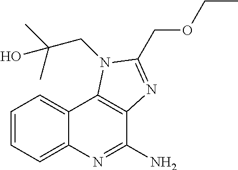 [SMILES: CCOCc1nc2c(N)nc3ccccc3c2n1CC(C)(C)O] |
| Imiquimod (R837) | 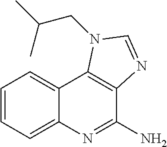 [SMILES: CC(C)Cn1cnc2c(N)nc3ccccc3c21] |
| CL264 | 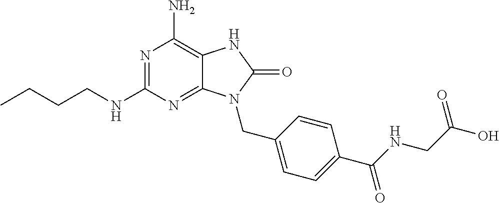 [SMILES: CCCCNc1nc(N)c2[nH]c(=O)n(Cc3ccc(C(=O)NCC(=O)O)cc3)c2n1] |
| Gardiquimod | 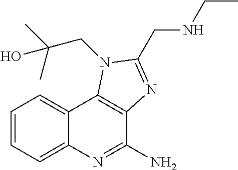 [SMILES: CCNCc1nc2c(N)nc3ccccc3c2n1CC(C)(C)O] |
| CL075 | 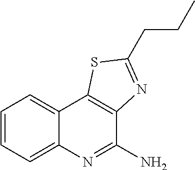 [SMILES: CCCc1nc2c(N)nc3ccccc3c2s1] |
| CL097 | 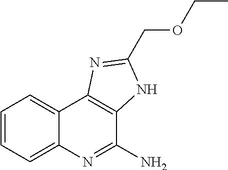 [SMILES: CCOCc1nc2c([nH]1)c(N)nc1ccccc12] |
| TL8-506 | 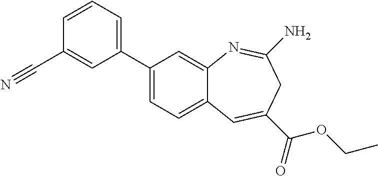 [SMILES: CCOC(=O)C1=Cc2ccc(-c3cccc(C#N)c3)cc2N=C(N)C1] |
| Telratolimod (3M-052) | 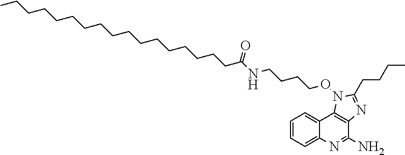 [SMILES: CCCCCCCCCCCCCCCCCC(=O)NCCCCOn1c(CCCC)nc2c(N)nc3ccccc3c21] |
| GSK2245035 | 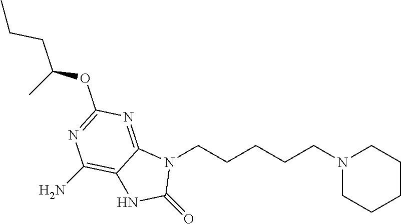 [SMILES: CCC[C@H](C)Oc1nc(N)c2[nH]c(=O)n(CCCCCN3CCCCC3)c2n1] |
| AZD8848 | 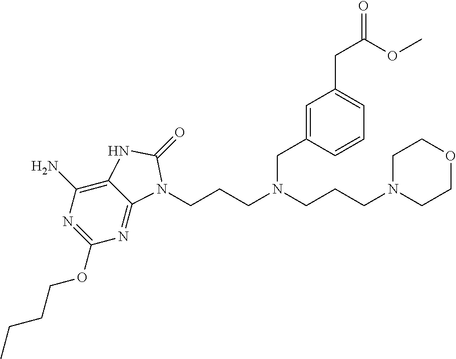 [SMILES: CCCCOc1nc(N)c2[nH]c(=O)n(CCCN(CCCN3CCOCC3)Cc3cccc(CC(=O)OC)c3)c2n1] |
| PF-4878691 | 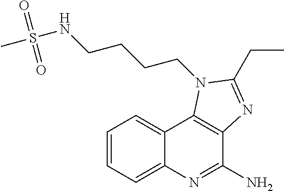 [SMILES: CCc1nc2c(N)nc3ccccc3c2n1CCCCNS(C)(=O)=O] |

TLR9, a pattern recognition receptor predominantly situated intracellularly within immune cells such as dendritic cells, macrophages, natural killer cells, and other antigen-presenting cells (APC). TLR9 agonists disclosed herein stimulate inflammatory cascades, leading to increased uptake and eradication of microorganisms and cancer cells, along with the elicitation of adaptive immune responses. Several TLR9 agonists have either obtained approval or progressed to the clinical stage. Examples include CpG 1018, licensed for use in Hepatitis B Virus (HBV) and COVID vaccines, as well as CpG 7909, employed in cancer treatment and HBV vaccine trials, among other applications.

In some embodiments, exemplary TLR9 agonists are described below:

| TLR9 agonists | sequence |
|---|---|
| ODN 1585 | 5′-ggGGTCAACGTTGAgggggg -3′ |
| | (SEQ ID NO: 1) |
| CpG 1018 | 5′-TGACTGTGAACGTTCGAGATGA-3′ |
| | (SEQ ID NO: 2) |
| ODN 1668 | 5′-TCCATGACGTTCCTGATGCT-3′ |
| | (SEQ ID NO: 3) |
| ODN 1826 | 5′-TCCATGACGTTCCTGACGTT-3′ |
| | (SEQ ID NO: 4) |
| ODN 2395 | 5′-TCGTCGTTTTCGGCGCGCGCCG-3′ |
| | (SEQ ID NO: 5) |
| ODN 2006 | 5′-TCGTCGTTTTGTCGTTTTGTCGTT-3′ |
| (ODN 7909) | (SEQ ID NO: 6) |
| ODN 2007 | 5′-CGTCGTTGTCGTTTTTGTCGTT-3′ |
| | (SEQ ID NO: 7) |
| ODN 10101 | 5′-TCGTCGTTTTCGCGCGCGCCG-3′ |
| | (SEQ ID NO: 8) |
| ODN D-SL01 | 5′-TCGCGACGTTCGCCCGACGTTCGGTA-3′ |
| | (SEQ ID NO: 9) |
| ODN D-SL03 | 5′-TCGCGAACGTTCGCCGCGTTCGAACGCGG-3′ |
| | (SEQ ID NO: 10) |

In some embodiments, the TLR agonist used in the adjuvant composition of the present disclosure is selected from TLR2 agonists, TLR3 agonists, TLR4 agonists, TLR7/8 agonists or TLR9 agonist described in the above tables.

### Fish Oil as Adjuvant

Fish oils, including squalene and squalane, are readily available from commercial sources or may be obtained by methods known in the art. Fish contain oils that can be easily extracted and utilized. Examples of such oils include cod liver oil, shark liver oils, and whale oil may be used in the present disclosure as adjuvants. In some embodiments, squalene and squalane (a saturated form of squalene), may be used and squalene is preferred. Squalene and squalane are commercially available or may be obtained by the methods known in the art.

In some embodiments, squalene or squalane may be used in a form of an oil (i.e., squalene or squalane) in water emulsion. The emulsion may comprise one or more additional oils. The additional oils may be tocopherols, such as α-, β-, γ-, δ-, ε- or ξ-tocopherols, among which α-tocopherols are preferred. In some embodiments, D-α-tocopherol and/or DL-α-tocopherol may be used, and DL-α-tocopherol is preferred.

### Vaccine Adjuvants

Despite the promising FDA approved adjuvants, there remains a need for new adjuvant system capable of increasing vaccine immunogenicity, durability, and reducing potential adverse effects. Moreover, this new adjuvant system exhibits good compatibility with various active ingredients. The present disclosure discloses novel adjuvant composition that strike a delicate balance between enhancing vaccine efficacy and ensuring safety, to address this need.

The inventors have found that the immunostimulatory activity of the saponin conjugate of Formula I or a pharmaceutically acceptable salt or solvate thereof can be increased by co-formulation with a TLR agonist. Accordingly, the present disclosure provides an inventive adjuvant composition (i.e., an inventive adjuvant system) comprising (i) a saponin conjugate of Formula I or a pharmaceutically acceptable salt or solvate thereof and (ii) a TLR agonist, which confer advantageous, and/or synergistic effects.

In some embodiments, the pharmaceutical combination of the present disclosure comprises a saponin conjugate of Formula I or a pharmaceutically acceptable salt or solvate thereof and a TLR agonist.

In some embodiments, the TLR agonist and the saponin conjugate are present in a weight ratio ranging from about 10:1 to about 1:100.

In some embodiments, the saponin conjugate or the pharmaceutically acceptable salt or solvate thereof of the present disclosure may be dissolved by solubilizing agents to increase solubility. These solubilizing agents may be used alone or in combination in an appropriate ratio. In the present disclosure, suitable solubilizing agents include, but are not limited thereto: citric acid, hydroxypropylcellulose, hydroxypropylmethylcellulose, sodium stearyl fumarate, methacrylic acid copolymer LD, methylcellulose, sodium lauryl sulfate, polyoxyl 40 stearate, purified shellac, sodium dehydroacetate, polysorbate 20, polysorbate 80, fumaric acid, DL-malic acid, L-ascorbyl stearate, L-asparagine acid, adipic acid, aminoalkyl methacrylate copolymer E, propylene glycol alginate, casein, casein sodium, a carboxyvinyl polymer, carboxymethylethylcellulose, powdered agar, guar gum, succinic acid, copolyvidone, cellulose acetate phthalate, tartaric acid, dioctylsodium sulfosuccinate, zein, powdered skim milk, sorbitan trioleate, lactic acid, aluminum lactate, ascorbyl palmitate, hydroxyethylmethylcellulose, hydroxypropylmethylcellulose acetate succinate, polyoxyethylene (105) polyoxypropylene (5) glycol, polyoxyethylene hydrogenated castor oil 60, polyoxyl 35 castor oil, poly(sodium 4-styrenesulfonate), polyvinyl acetal diethylamino acetate, polyvinyl alcohol, maleic acid, methacrylic acid copolymer S, lauromacrogol, sulfuric acid, aluminum sulfate, phosphoric acid, calcium dihydrogen phosphate, sodium dodecylbenzenesulfonate, a vinyl pyrrolidone-vinyl acetate copolymer, sodium lauroyl sarcosinate, acetyl tryptophan, sodium methyl sulfate, sodium ethyl sulfate, sodium butyl sulfate, sodium octyl sulfate, sodium decyl sulfate, sodium tetradecyl sulfate, sodium hexadecyl sulfate and sodium octadecyl sulfate.

In some embodiments, the saponin conjugate of Formula I or a pharmaceutically acceptable salt or solvate thereof and the TLR adjuvant are encapsulated in liposomes. In some embodiments, one or more liposome-forming compounds may be used for the preparation of liposomes. The liposome-forming compounds may be selected from the group consisting of phospholipids, such as 1,2-dioleoyl-sn-glycero-3-phosphocholine (DOPC), 1,2-dimyristoyl-sn-glycero-3-phosphocholine (DMPC), dioleoyl phosphatidylglycerol (DOPG), dimyristoyl phosphatidylglycerol (DMPG), 1,2-dioleoyloxy-3-(trimethylammonium)propane (DOTAP)), cholesterol and the combination thereof.

In some embodiments, the saponin conjugate of Formula I or a pharmaceutically acceptable salt or solvate thereof are in the form of an oil in water emulsion. In some embodiments, one or more emulsion-forming compounds may improve the stability and immune-stimulating properties of the adjuvant composition. The emulsion-forming compounds may be selected from the group consisting of polysorbate, α-tocopherol, span 85, glycerol, poloxamer 188 and carboxymethyl cellulose and the combination thereof.

While the exact mechanism of adjuvants is not entirely clear, there are reports suggesting a connection with dendritic cells. In some embodiments, to enhance the uptake of adjuvants by dendritic cells, the liposomes produced may have a particle size smaller than 250 nm (e.g., 220 nm, 190 nm, 180 nm, 170 nm, 160 nm, 150 nm, 120 nm, 100 nm or less). In some embodiments, lipid combinations, including phospholipids, cholesterol (chol), and combinations of these lipids, are used for encapsulating the saponin and TLR agonists. In some embodiments, the particle size of liposomes can be analyzed by transmission electron microscopy (TEM) and/or Zetasizer Nano, Malvern.

### Pharmaceutical Composition Comprising the Vaccine Composition

The present disclosure also provides pharmaceutical compositions comprising an antigen and an inventive adjuvant composition comprising a Synsap or a pharmaceutically acceptable salt or solvate thereof in combination with a TLR agonist. In some embodiments, the pharmaceutical compositions are vaccine compositions. Any animal that may experience the beneficial effects of the compositions of the present disclosure within the scope of subjects that may be treated or administered with the compositions or combinations of the present disclosure. In some embodiments, the subjects are mammals. In some embodiments, the subjects are humans.

In some embodiments, the amount of antigen in the pharmaceutical compositions may vary depending on the species of the antigen and so on. In some embodiments, each does may comprises 1-1000 μg of antigen, for example, 1 μg, 2 μg, 5 μg, 10 μg, 15 μg, 20 μg, 25 μg, 30 μg, 40 μg, 50 μg, 60 μg, 100 μg, 200 μg, 300 μg, 400 μg, 500 μg, 600 μg, 800 μg or 1000 μg. However, the present disclosure is not limited thereto.

In some embodiments, the pharmaceutical composition of the present disclosure may further comprise a pharmaceutically acceptable excipient. In some embodiments, the pharmaceutical compositions of the present disclosure may further comprise one or more of wetting agents, emulsifiers and lubricants, such as sodium lauryl sulfate and magnesium stearate, as well as coloring agents, release agents, coating agents, sweetening flavoring and perfuming agents, preservatives and antioxidants.

The administration of the vaccine (or the antisera which it elicits) may be for either a “prophylactic” or “therapeutic” purpose. The prophylactic administration of the vaccine(s) serves to prevent or attenuate any subsequent presentation of the disease. When provided prophylactically, the vaccine(s) are provided in advance of any symptoms of disease. When provided therapeutically, the vaccine(s) is provided upon or after the detection of symptoms which indicate that an animal may be infected with a pathogen or have a certain cancer. The therapeutic administration of the vaccine(s) serves to attenuate any actual disease presentation. Thus, the vaccines may be provided either prior to the onset of disease proliferation or after the initiation of an actual proliferation.

Thus, in one aspect the present disclosure provides vaccines comprising one or more bacterial, viral, protozoal, or tumor-related antigens in combination with the inventive adjuvant of the present disclosure. In some embodiments, the vaccine comprises a single bacterial, protozoal, viral, or tumor-related antigen in combination with the inventive adjuvant of the present disclosure.

In some embodiments, one or more antigens of provided vaccines are bacterial antigens. In certain embodiments, the bacterial antigens are antigens associated with a bacterium selected from the group consisting of Bordetella pertussis, Bordetella parapertussis, Bordetella bronchiseptica, Borrelia burgdorferi, Borrelia spp., Chlamydia trachomatis, Helicobacter pylori, Chlamydia pneumoniae, Ureaplasma urealyticum, Mycoplasma pneumoniae, Staphylococcus spp., Staphylococcus aureus, Streptococcus pyogenes, Streptococcus spp., Streptococcus pneumoniae, Streptococcus viridans, Enterococcus faecalis, Neisseria meningitidis, Neisseria gonorrhoeae, Bacillus anthracis, Salmonella spp., Salmonella typhi, Vibrio cholera, Pasteurella pestis, Campylobacter spp., Campylobacter jejuni, Clostridium spp., Clostridium difficile, Corynebacterium diphtheria, Mycobacterium spp., Mycobacterium tuberculosis, Pseudomonas aeruginosa, Treponema spp., Leptospria spp., Hemophilus ducreyi, Hemophilus influenza, Escherichia coli, Shigella spp., Ehrlichia spp., Rickettsia spp. and combinations thereof.

In certain embodiments, one or more antigens of provided vaccines are viral-associated antigens. In certain embodiments, the viral-associated antigens are antigens associated with a virus selected from the group consisting of influenza viruses, parainfluenza viruses, mumps virus, adenoviruses, respiratory syncytial virus, Epstein-Barr virus, rhinoviruses, polioviruses, coxsackieviruses, echo viruses, rubeola virus, rubella virus, varicella-zoster virus, herpes viruses, herpes simplex virus, herpes type 1 to type 8, parvoviruses, cytomegalovirus, hepatitis viruses, human papillomavirus, alphaviruses, flaviviruses, bunyaviruses, rabies virus, arenaviruses, filoviruses, HIV 1, HIV 2, HTLV-1, HTLV-II, FeLV, bovine LV, FeIV, canine distemper virus, canine contagious hepatitis virus, feline calicivirus, feline rhinotracheitis virus, TGE virus, foot and mouth disease virus, coronavirus, dengue virus, Flavivirus and combinations thereof.

In certain embodiments, one or more antigens of provided vaccines are tumor-associated antigens. In some embodiments, the tumor-associated antigens are antigens selected from the group consisting of killed tumor cells and lysates thereof, MAGE-1, MAGE-3 and peptide fragments thereof, human chorionic gonadotropin and peptide fragments thereof, carcinoembryonic antigen and peptide fragments thereof, alpha fetoprotein and peptide fragments thereof; pancreatic oncofetal antigen and peptide fragments thereof; prostate-specific antigens and peptide fragments thereof; MUC-1 and peptide fragments thereof, CA 125, CA 15-3, CA 19-9, CA 549, CA 195 and peptide fragments thereof, prostate-specific membrane antigen and peptide fragments thereof, squamous cell carcinoma antigen and peptide fragments thereof, ovarian cancer antigen and peptide fragments thereof, pancreas cancer associated antigen and peptide fragments thereof; Her1/neu and peptide fragments thereof, gp-100 and peptide fragments thereof; mutant K-Ras proteins and peptide fragments thereof; mutant p53 and peptide fragments thereof, truncated epidermal growth factor receptor, chimeric protein p210^{BCR-ABL}, STn, Tn, Lewis^{x}, Lewis^{y}, TF, GM1, GM2, GD2, GD3, Gb3, KH-1, Globo-H, SSEA-4; and mixtures thereof.

As described above, the adjuvant of the present disclosure may be used in cancer vaccines as adjuvant in combination with tumor-associated antigens. In certain embodiments, vaccines may be used in the treatment or prevention of tumors. In certain embodiments, the tumor is a benign neoplasm. In other embodiments, the tumor is a malignant neoplasm.

Another aspect of the present disclosure relates to methods for immunizing a subject with the vaccine composition of the present disclosure.

The pharmaceutical compositions of the present disclosure may be prepared in various forms. For example, they can be formulated as injectables, either as liquid solutions or suspensions. Solid forms that are suitable for reconstitution into solution, or suspension in liquid vehicles prior to injection can also be prepared (e.g., lyophilized or spray-lyophilized compositions), although aqueous compositions are preferred. Suspensions for intramuscular or intradermal or subcutaneous injections are typical.

The present disclosure provides a method of raising an immune response in a subject, comprising administering to the subject a composition or combination as described herein.

The immune response stimulated by the vaccine as described herein generally includes an antibody response, preferably a protective antibody response. An immune response can also include a cellular response. Methods are well known in the art for assessing antibody and cellular immune responses following immunization.

### Definitions

As used herein, the following definitions shall apply unless otherwise indicated.

“Stereoisomer” or “stereoisomers” refer to compounds that differ in the chirality of one or more stereocenters. Stereoisomers include enantiomers and diastereomers.

“Subject” refers to mammals and includes humans and non-human mammals.

The term “aliphatic” or “aliphatic group” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle”, “cycloaliphatic” or “cycloalkyl”) that has a single point of attachment to the rest of the molecule. Unless otherwise specified, aliphatic groups contain aliphatic carbon atoms. In some embodiments, aliphatic groups contain 1-20 aliphatic carbon atoms. In some embodiments, aliphatic groups contain 1-12 aliphatic carbon atoms. In some embodiments, aliphatic groups contain 1-11 aliphatic carbon atoms. In other embodiments, aliphatic groups contain 1-10 aliphatic carbon atoms. In still other embodiments, aliphatic groups contain 1-9 aliphatic carbon atoms, and in yet other embodiments, aliphatic groups contain 1-8 aliphatic carbon atoms. In still other embodiments, aliphatic groups contain 1-7 aliphatic carbon atoms. In other embodiments, aliphatic groups contain 1-6 aliphatic carbon atoms. In other embodiments, aliphatic groups contain 1-5 aliphatic carbon atoms. In other embodiments, aliphatic groups contain 1-4 aliphatic carbon atoms. In other embodiments, aliphatic groups contain 1-3 aliphatic carbon atoms, and in yet other embodiments, aliphatic groups contain 1-2 aliphatic carbon atoms. Suitable aliphatic groups include, but not limited to, linear or branched, substituted or unsubstituted alkyl, alkenyl, alkynyl groups and hybrids or combinations thereof.

In some embodiments, cycloaliphatic (or “carbocycle” or “cycloalkyl”) refers to a monocyclic C—C hydrocarbon that is completely saturated or that contains one or more units of unsaturation, but which is not aromatic, that has a single point of attachment to the rest of the molecule. Suitable cycloaliphatic groups include, but are not limited to, cycloalkyl, cycloalkenyl, cycloalkynyl, (cycloalkyl)alkyl, (cycloalkenyl)alkyl, (cycloalkynyl)alkyl, (cycloalkyl)alkenyl, (cycloalkenyl)alkenyl. (cycloalkynyl)alkenyl, (cycloalkyl)alkynyl, (cycloalkenyl)alkenyl or (cycloalkynyl)alkynyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or a substitutable nitrogen of a heterocyclic ring, for example N (as in 3,4-dihydro-2H-pyrrolyl), NH (as in pyrrolidinyl) or NR″ (as in N-substituted pyrrolidinyl)).

The term “unsaturated” as used herein, means that a moiety has one or more double bond(s) or triple bond(s).

The term “halogen” means F, Cl, Br, or I.

The term “acyl” used alone or a part of a larger moiety, refers to groups formed by removing a hydroxy group from a carboxylic acid.

The terms “arylkyl” and “arylaliphatic” are used interchangeably and refer to aliphatic groups in which a hydrogen atom has been replaced with an aryl group. Such aryl groups include, without limitation, phenyl, biphenyl, naphthyl, cinnamyl and dihyrocinnamyl.

The term “aryl” used alone or as part of a larger moiety as in “aryl-aliphatic”. The term “aryl” refers to mono-cyclic or multi-cyclic, monovalent aromatic radical. In some embodiments, an aryl has 6 to 20 (C6-20), 6 to 15 (C6-15), 6 to 12 (C6-12) or 6 to 10 (C6-10) ring atoms. In certain embodiments of the present disclosure, “aryl” refers to an aromatic ring system which includes, but not limited to, benzyl, phenyl, biphenyl, naphthyl, anthracyl and the like, which may bear one or more substituents. Also included within the scope of the term “aryl”, as it is used herein, is a group in which an aromatic ring is fused to none or one or more non-aromatic rings, such as indanyl, phthalimidyl, naphthimidyl, phenanthridinyl, or tetrahydronaphthyl, and the like. The term “aryl” may be used interchangeably with the term “aryl ring.”

The term aryloxy-aliphatic (or “aralkoxy”, or “arylkoxy”, or “aryloxyalkyl”) refers to monocyclic or bicyclic ring systems having a total of five to fourteen ring members, wherein at least one ring in the system is an aromatic ring and wherein each ring in the system contains 3 to 7 ring members.

The terms “heteroaryl” used alone or as part of a larger moiety, e.g., “heteroaryloxy” or “heteroaryl-apliphatic”, or “heteroarylkyl” refer to groups having 4 to 10 ring atoms, preferably 4, 5, 6, or 9 ring atoms; having 6, 10, or 14 electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl pyrazolyl, triazolyl, tetrazolyl, oxazolyl, isoxazolyl, oxadiazolyl, thiazolyl, isothiazolyl, thiadiazolyl pyridyl, pyridazinyl, pyrimidinyl, pyrazinyl, indolizinyl, purinyl, naphthyridinyl, and pteridinyl.

The terms “heteroaryl” and “heteroar-”, as used herein, also include groups in which a heteroaromatic ring is fused to one or more aryl, cycloaliphatic, or heterocyclyl rings, where the point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and 2H-pyrido [2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic” and any of which terms include rings that are optionally substituted. The terms “heteroaryl-aliphatic” and “heteroaryl-alkyl” refer to an aliphatic group substituted by a heteroaryl moiety, wherein the aliphatic and heteroaryl portions independently are optionally substituted.

The term “hetero-aliphatic” as used herein, means aliphatic groups wherein one or two carbon atoms are independently replaced by one or more of oxygen, sulfur, nitrogen, or phosphorous. Hetero-aliphatic groups may be substituted or unsubstituted, branched or unbranched, cyclic or acyclic, and include “heterocycle”, “heterocyclyl”, “heterocycloaliphatic”, or “heterocyclic” groups.

As used herein, the terms “heterocycle”, “heterocyclyl”, and “heterocyclic ring are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl.

The terms “heterocycle”, “heterocyclyl”, “heterocyclyl ring”, “heterocyclic group”, and “heterocyclic moiety” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono or bicyclic.

The term “heterocyclylaliphatic” refers to an alkyl group substituted by a heterocyclyl, wherein the aliphatic group and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

In another aspect, the present disclosure provides “pharmaceutically acceptable” compositions, which comprises a therapeutically effective amount of one or more of the compounds described herein, formulated together with one or more pharmaceutically acceptable carriers (additives) and/or diluents. As described in detail, the pharmaceutical compositions of the present disclosure may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by intramuscular, subcutaneous, intravenous or epidural injection as, for example, a sterile solution or suspension, or sustained release formulation; topical application, for example, as a cream, ointment, or a controlled-release spray or patch applied to the lungs, skin or oral cavity; intravaginally or intrarectally, for example, as a pessary, cream or foam; sublingually: ocularly; transdermally; or nasally, pulmonary and to other mucosal surfaces.

The phrase “pharmaceutically acceptable” is employed herein to refer to those compounds, compositions, materials, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition or vehicle. Such as a liquid or solid filler, excipient, diluent, or solvent encapsulating material, involved in transporting or carrying the subject compound from one portion of the body to another portion of the body. Each carrier must be “acceptable” in the sense of being compatible with the other ingredients of the formulation and not injurious to the patient. Some examples of materials which can serve as pharmaceutically-acceptable carriers include: sugars, such as glucose, lactose, sucrose; starches, such as corn starch and potato starch; cellulose, and its derivatives, such as sodium carboxymethyl cellulose, ethyl cellulose and cellulose acetate; powdered tragacanth; malt, gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols. Such as glycerin, sorbitol, mannitol and polyethylene glycol; esters, such as ethyl oleate and ethyl laurate, agar, buffering agents, such as magnesium hydroxide and aluminum hydroxide; alginic acid; pyrogen-free water, isotonic saline: Ringer's solution: ethyl alcohol; pH buffered solutions; polyesters, polycarbonates and/or polyanhydrides; and other non-toxic compatible substances employed in pharmaceutical formulations.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are technically well known in the art. For example, S. M. Berge et al. describes pharmaceutically acceptable salts in detail in. J. Pharmaceutical Sciences, 1977, 66, 1-19, incorporated herein by reference. Pharmaceutically acceptable salts of the compounds of this present disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyethanesulfonate, lactobionate, lactate, laurate, lauryl sulfate, malate, maleate, malonate, methanesulfonate, 2-naphthale nesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

In some cases, the compounds of the present disclosure may be capable of forming pharmaceutically acceptable salts with inorganic or organic acids or with inorganic or organic base. The term “pharmaceutically acceptable salts” in these instances may refer to the relatively non-toxic, inorganic and organic base addition salts of compounds of the present disclosure, or the relatively non-toxic, inorganic and organic acid addition salts of compounds of the present disclosure. These salts can likewise be prepared in situ in the administration vehicle or the dosage form manufacturing process, or by separately reacting to the purified compound with a suitable base or acid.

In some cases, the compounds of the present disclosure or pharmaceutical acceptable salts thereof may be capable of forming pharmaceutically acceptable solvates. The term “solvate” means solvent addition forms that contain either stoichiometric or non-stoichiometric amounts of solvent. Some compounds tend to trap solvent molecules, thus forming a solvate. The term “hydrate” is employed when the solvent is water and for the avoidance of any doubt, the term “hydrate” is encompassed by the term “solvate”. Examples of hydrates include monohydrates, dihydrates, but are not limited thereto. Examples of solvates include ethanol solvates, acetone solvates but is not limited thereto.

Unless otherwise stated, structures depicted herein are also meant to include all isomeric (e.g., enantiomeric, diastereomeric, and geometric, conformational forms of the structure; for example, the R and S configurations for each stereocenter, cis and trans double bond isomers, and Z and E conformational isomers. Therefore, single stereochemical isomers as well as enantiomeric, diastereomeric, and geometric (or conformational) mixtures of the present compounds are within the scope of the present disclosure. Unless otherwise stated, all tautomeric forms of the compounds of the present disclosure are within the scope of the present disclosure.

Provided compounds may comprise one or more saccharide moieties. Unless otherwise specified, both D- and L-configurations, and mixtures thereof, are within the scope of the present disclosure. Unless otherwise specified, both C- and S-linked embodiments, and mixtures thereof, are contemplated by the present disclosure.

Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a 13C- or 14C-enriched carbon are within the scope of the present disclosure. Such compounds are useful, for example, as analytical tools, as probes in biological assays, or as therapeutic agents in accordance with the present disclosure.

According to embodiments of the present disclosure, the phrase “protecting group” as used herein means temporary modifications of a potentially reactive functional group which protect it from undesired chemical transformations. Examples of such protecting groups include esters of carboxylic acids, silyl ethers of alcohols, and acetals and ketals of aldehydes and ketones, respectively. Of course, other appropriate protection groups may be used. Additionally, a variety of protecting groups are described by Greene and Wuts (supra).

Furthermore, in one embodiment, the present disclosure provides a method for stimulating, inhibiting, suppressing or modulating an immune response in a subject. The method may include, inter alia, administering to a subject any one of the compounds of this present disclosure or any combination thereof.

Furthermore, in one embodiment, the present disclosure provides a method for stimulating, inhibiting, suppressing or modulating an immune response in a subject. The method includes, inter alia, administering to a subject a pharmaceutical composition including, inter alia, any one of the compounds of this disclosure or any combination thereof, together with one or more pharmaceutically acceptable excipients.

Furthermore, in one embodiment, “pharmaceutical composition” can mean a therapeutically effective amount of one or more compounds of the present disclosure together with suitable excipients and/or carriers useful for stimulating, inhibiting, suppressing or modulating an immune response in a subject.

In one embodiment, “therapeutically effective amount” may refer to that amount that provides a therapeutic effect for a given condition and administration regimen. In one embodiment, such compositions can be administered by any method known in the art.

As described herein, compounds of the present disclosure may contain “optionally substituted” moieties. In general, the term “substituted”, whether preceded by the term “optionally’ or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by the present disclosure are preferably those that resulted in the formation of stable or chemically feasible compounds.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration”, “administered systemically”, “peripheral administration” and “administered peripherally” as used herein mean the administration of a compound, drug or other material other than directly into the central nervous system, such that it enters the patient's system and, thus, is subjected to metabolism and other like processes, for example, subcutaneous administration.

The term “pure” refers to compounds that are substantially free of compounds of related non-target structure or chemical precursors (when chemically synthesized). This quality may be measured or expressed as “purity.” In some embodiments, a target compound has less than about 30%, 20%, 10%, 5%, 2%, 1%, 0.5%, and 0.1% of non-target structures or chemical precursors.

The term “carbohydrate” refers to a sugar or polymer of sugars. The terms “saccharide” and “carbohydrate” may be used interchangeably. Most carbohydrates are aldehydes or ketones with many hydroxyl groups, usually one on each carbon atom of the molecule. Carbohydrates generally have the molecular formula CnH2nOn. A carbohydrate may be monosaccharide, a disaccharide, trisaccharide, oligosaccharide, or polysaccharide. The most basic carbohydrate is monosaccharide, such as glucose, galactose, sucrose, ribose, mannose, arabinose, xylose, and fructose. Disaccharides are two joined monosaccharides. Exemplary disaccharides include sucrose, lactose, cellobiose, and maltose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose. Carbohydrates may exist in many different forms, for example, conformers, cyclic forms, acyclic forms, stereoisomers, tautomers, anomers, and isomers.

The term “liposomes” refers to closed bilayer membranes containing an entrapped aqueous volume. Liposomes can manifest as either unilamellar vesicles characterized by a singular membrane bilayer or multi-lamellar vesicles featuring multiple membrane bilayers, each delineated by an intervening aqueous layer. The structure of the resulting membrane bilayer is such that the hydrophobic (non-polar) tails of the lipid are oriented toward the center of the bilayer while the hydrophilic (polar) heads orient towards the aqueous phase. Liposomes, as they are ordinarily used, consist of smectic mesophases, and can consist of either phospholipid or non-phospholipid smectic mesophases.

An adjuvant formulation, designated as AS01 (also referred to as AS01B or AS01E), was previously disclosed by GlaxoSmithKline. The lipid bilayer of AS01 comprises a neutral lipid that remains in a non-crystalline state at room temperature. In some respects, the lipid bilayer includes dioleoyl phosphatidylcholine, cholesterol, monophosphoryl lipid A (MPLA), and QS-21. Additional details regarding such formulations are provided in U.S. Published Patent Application No. 2011/0206758. During the manufacturing process of AS01, small unilamellar liposomal vesicles (SUVs) are initially formed, followed by the addition of purified QS-21 to the SUVs. QS-21 exhibits unique properties by interacting with liposomal cholesterol, leading to the formation of perforations or other permanent structural modifications within the liposomes. See, for example, Alving et al., Front Immunol 2023 Vol. 14 Pages 1102524. It is presumed that a reduction in free QS-21 levels results in decreased local injection site pain, which is often associated with the presence of free QS-21.

In the embodiments of present disclosure, the adjuvant composition comprises a liposomal composition containing a TLR4 agonist and at least one Synsap, wherein the Synsap is not soluble in water and therefore needs to be encapsulated within the liposome or externally associated with the formulation, wherein the liposome comprises i) a lipid bilayer comprising phospholipid (e.g., 1,2-dimyristoyl-sn-glycero-3-phosphocholine (DMPC) and/or dimyristoyl phosphatidylglycerol (DMPG) and/or 1,2-dioleoyl-sn-glycero-3-phosphocholine (DOPC) and/or dioleoyl phosphatidylglycerol (DOPG) and/or 1,2-dioleoyloxy-3-(trimethylammonium)propane (DOTAP) and ii) cholesterol and iii) When Synsap is encapsulated within the liposome, its mole percent concentration is less than about 9% (mol/mol), preferably about 2% to 5%, and more preferably about 3.8%; however, when Synsap is externally added to the liposome, a solubilizing agent is required for it to dissolve in the aqueous solution. It was noteworthy that this solubilizing agent would not damage the liposome formulation. Moreover, unlike QS-21, Synsap does not cause hemolysis, making it highly safe. At least these three features are distinct from those of AS01 as discussed above. The adjuvant composition may contain about 2 mg or less, about 1.5 mg or less, 1 mg or less, 0.9 mg or less, 0.8 mg or less, 0.7 mg or less, 0.6 mg or less, 0.5 mg or less, 0.4 mg or less, 0.3 mg or less, 0.2 mg or less, 0.1 mg or less of Synsap per mL liposome suspension.

As used herein, the singular form “a”, “an” and “the” include plural references unless the context clearly dictates otherwise. The numerical range (such as 10% to 11% of A) includes the upper and lower limits (i.e., 10%≤A≤11%) unless otherwise specified. If the numerical range does not define the lower limit (such as less than 0.2% of B, or below 0.2% of B), it means that the lower limit may be 0 (i.e., 0%≤B≤0.2%). The above terms are made for the purpose of describing and illustrating the present disclosure and should not be taken in a limiting sense.

The pharmaceutically acceptable carrier may include one or more agents selected from the group consisting of solvents, stabilizers, emulsifiers, suspending agents, decomposing agents, flavoring agents, binding agents, excipients, cosolvents, chelating agents, diluents, gelling agents, preservatives, lubricants and surfactants.

The term “administering” the composition to a subject refers to directly administering the composition to the subject, and the composition can be administered by professional medical personnel, or on the subject's own.

The term “cancer” herein includes the broader term “abnormal cell proliferation”, which is also called “excessive cell proliferation” or “proliferative disease”. Examples of diseases associated with abnormal cell proliferation include metastatic tumors, malignant tumors, benign tumors, cancers, precancerous lesions, hyperplasia, and polyps.

The term “treatment” refers to methods used to obtain beneficial or desired results (including clinical results). For the purpose of the present disclosure, beneficial or desired clinical results include, but are not limited to, reduction or improvement of one or more symptoms, reduction of the disease degree, stability of the disease state (i.e., no deterioration), prevention of the disease spread, delay or slowing of the disease progress, and improvement, alleviation and remission (partial or complete remission) of the disease state.

The term “treatment” may also mean prolonged survival period if compared with the expected survival period without treatment.

In the following experiments, the experimental methods without specific conditions are selected according to conventional methods and conditions, or according to the instructions of the kit.

### Abbreviation

Synsap: synthetic saponin.
DOPC: 1,2-dioleoyl-sn-glycero-3-phosphocholine
DMPC: 1,2-dimyristoyl-sn-glycero-3-phosphocholine
DOPG: dioleoyl phosphatidylglycerol
DMPG: dimyristoyl phosphatidylglycerol
DOTAP: 1,2-dioleoyloxy-3-(trimethylammonium)propane
Chol: cholesterol
MPL: monophosphoryl lipid
TPGS: D-α-tocopheryl polyethylene glycol succinate

### Materials and Methods

### Preparation of Liposomes

Lipids were dissolved in solvent such as methanol, ethanol and chloroform. A lipid film was formed by the solvent evaporation under vacuum. PBS solution was added to the mixture which was then subjected to pre-homogenization followed by high pressure homogenization at 20,000 psi. The resulting liposomes were sterile filtered through a 0.22 mm membrane and distributed into glass containers. DLS analysis showed that the liposome maintained high uniformity (PdI <0.3) and a particle size ranged from 50-200 nm. When Synsap was additionally added to the liposome solution, 2 mg of Synsap was dissolved in 3 mg of TPGS (D-α-tocopheryl polyethylene glycol succinate), 45 mg of propylene glycol and 45 mg of ethanol, followed by dilution with 2 mL of PBS solution, pH 7.2.

### Preparation of Oil in Water Emulsion

49 mg of polysorbate 80 was dissolved in PBS solution. Separately, 5.0 mg of SynSap, 0.5 mg of TLR4 agonist, and 119 mg of α-tocopherol were dissolved in 107 mg of squalene. The two solutions were then combined, and the mixture underwent pre-homogenization followed by high-pressure homogenization at 20,000 psi for approximately 10 to 15 cycles. The resulting liposomes were sterile-filtered through a 0.22 m membrane and distributed into glass containers. DLS analysis showed that the liposomes maintained high uniformity (PdI <0.3) with a particle size ranging from 50 to 200 nm.

### Immunization

Mice were immunized intramuscularly (right leg quadriceps femoris muscle) with 30 G syringes (BD Ultra-Fine Insulin Syringes 30 G) containing 100 μL of vaccines on days 0 and 14. The adjuvant formulations were prepared as mentioned in Example 1-Example 5. H3N2 antigen was obtained from Adimmune, and was mixed with the adjuvant formulation before injecting to mice. Body weights were measured at pre-dose (D0), D1, D2, D14, D15, D16 and D21.

### Biological Sampling

Blood was collected via submandibular bleed at the specific time point mentioned in Example. Terminal blood was collected into a 1 ml 27 G syringe by cardiac puncture. Followed by centrifugation at 10,000 g for 5 minutes, at 25° C. Supernatant was aliquoted into a microcentrifuge tube and stored at −20° C. freezer.

### IgG ELISA

Serum anti-H3N2 IgG antibody titers were measured using an ELISA. Maxisorp plates were coated with whole inactivated H3N2 antigen at 2 mcg/mL in 100 mM of carbonate buffer overnight at 4° C. Plates were blocked with blocking buffer (1% BSA in PBS) for at least 1 h at 25° C. and then were washed with 0.05% PBST. Serial 10-fold dilutions of serum samples were added to plates. After 1 h, plates were washed with 0.05% PBST and incubated with 10-fold dilutions of mouse sera. After 1 h, plates were washed with 0.05% PBST Tween 20 and secondary antibody was added. All peroxide labelled goat Anti-Mouse IgG (1:5000, Southernbiotech), goat Anti-Mouse IgG1 (1:5000, Southernbiotech), and goat anti-mouse IgG2c (1:5000, Southernbiotech) were diluted in 1% BSA-PBS were added separately for 1 h. The plates were washed with 0.05% PBST and were developed with TMB Chromogen Solution (Invitrogen, Cat. no. 00-2023) for 15 min, followed with stop solution (1 N HCl). The OD450 values were read by Spectramax 190 (Molecular Devices).

### Hemagglutination Inhibition Test

Serum was also tested for the presence of functional antibodies by hemagglutination inhibition (HAI) according to WHO standard protocols with minor modifications. The sera were diluted 1:5 in ROE (ROE (II) and placed in 37° C. water bath overnight (18-20 hours). Sera were heat-inactivated at 56° C. for 30 minutes. An additional 1:2 dilution with PBS was performed, leading to a final serum dilution of 1:10. Guinea pig red blood cells (GRBC) were prepared by mixing 0.75% GRBC in PBS/0.75% BSA. Each well of a 96-well U bottom assay plate was filled with 25 μI PBS. Sera were added across the top row and diluted down the columns in two-fold dilutions.

Each sample was tested in duplicate. The second to last column contained the positive control sera and the last column contained the negative control (PBS) and the virus back-titration. 25 μL of virus was added to each well except the last column. The plates were agitated and incubated for 1 hour at room temperature. 50 μL of GRBC were then added to each well, followed by a 1-hour incubation, after which the hemagglutination patterns were read by observed as a “halo” or circle of settled cells in the bottom of the wells.

### Intracellular Staining Flow Cytometry

Splenocytes were seeded on 96-well plate at 2,000,000 cell/0.2 mL. Splenocytes were stimulated with or without antigen (0.2 mcg H3N2/well) and cultivated 2 h in a CO₂ incubator, and cells were treated with protein transport inhibitor Brefeldin (Invitrogen, Cat. no. 00-4506-51) for another 12 hours at 37° C. Afterward, cells were harvested then washed twice with PBS and then stained for viability dye (eBioscience, Cat. 65-0866-14) and surface CD3 (BioLegend, Cat. 100240), CD4 (BioLegend, Cat. 100540), CD8 (eBioscience, Cat. 11-0081-82), CD44 (BioLegend, Cat. 103007), CD62L (BioLegend, Cat. 104412) for 30 minutes at 4° C. After washing, cells were fixed for 30 minutes at room temperature using IC fixation buffer (Invitrogen, Cat. 00-8222-49), then cells were washed in permeabilization buffer (Invitrogen, Cat. 00-8333-56) and stained with IFNγ antibody (BioLegend, Cat. no. 505824), and IL-2 antibody (eBioscience, Cat. no. 25-7021-82). The cells were acquired on a Attune Nxt (Thermo Fisher Scientific) and analyzed with Flowjo software (v 10).

### Enzyme-Linked Immunosorbent Assay for IFNγ and IL-4

Spleen tissue was isolated from mice and processed into single-cell suspensions using a microcentrifuge sample pestle. Splenocytes were seeded on 96 well plates at 2,000,000/0.2 mL, stimulated with or without the 2 mcg/mL H3N2, and cultivated for 40 hours in a CO₂ incubator at 37° C. For detection of IFNγ, and IL-4, the culture supernatant from the 96-well microplates was harvested to analyze the levels of cytokines by ELISA using ELISA MAX™ Deluxe Set Mouse IFNγ (Biolegend, Cat. No. 430816), and IL-4 (Biolegend, Cat. No. 431104). The OD450 values were read by Spectramax 190 (Molecular Devices).

### Statistical Analysis

Statistical analyses were performed using GraphPad Prism. Data were expressed as mean±standard error (SEM) values. For antibody subtype quantification, a sigmoidal 4-parameter nonlinear regression curve fit was used. Statistical comparisons between multiple groups were analyzed by the student's t-test or one-way ANOVA test, followed by Tukey's multiple comparison test. A p-value <0.05 was considered statistically significant.

### Example 1: Preparation of the Vaccine Composition (Synsap/TLR4 Agonist)

### Example 1-1 to Example 1-4

The DOPC, cholesterol, PHAD and Synsap 3 were mixed and dissolved in methanol. Subsequently, Subsequently, the liposomes were prepared according to the section of the preparation of liposomes as mentioned previously. A vaccine composition was prepared by combining a 5 mcg dose of PHAD (from Avanti) with either 5 mcg, or 50 mcg, 75 mcg or 100 mcg of Synsap 3 as the adjuvant, along with 0.3 mcg of H3N2.

### Example 1-5 to Example 1-8

The DOPC, cholesterol, 3D-PHAD and Synsap 3 were mixed and dissolved in methanol. Subsequently, the liposome was prepared according to the section of the preparation of liposomes as mentioned previously. A vaccine composition was prepared by combining a 5 mcg dose of 3D-PHAD (from Avanti) with either 5 mcg, 50 mcg, 75 mcg or 100 mcg of Synsap 3 as the adjuvant, along with 0.3 mcg of H3N2.

### Example 1-9 to Example 1-13

The DOPC, cholesterol, 3D-6A-PHAD and Synsap 3 were mixed and dissolved in methanol. Subsequently, the liposome was prepared according to the section of the preparation of liposomes as mentioned previously. A vaccine composition was prepared by combining a 1 mcg dose of 3D-6A-PHAD (from Avanti) with either 5 mcg, 50 mcg, 75 mcg or 100 mcg of Synsap 3 as the adjuvant, along with 0.3 mcg of H3N2. Another vaccine composition was prepared by combining a 5 mcg dose of 3D-6A-PHAD (from Avanti) with 50 mcg of Synsap 3 as the adjuvant, along with 0.3 mcg of H3N2.

### Example 1-14 to Example 1-18

The DOPC, cholesterol, EcML and Synsap 3 were mixed and dissolved in methanol. Subsequently, the liposome was prepared according to the section of the preparation of liposomes as mentioned previously. A vaccine composition was prepared by combining a 1 mcg dose of EcML with either 5 mcg, 50 mcg, or 75 mcg or 100 mcg of Synsap 3 as the adjuvant, along with 0.3 mcg of H3N2. Another vaccine composition was prepared by combining a 5 mcg dose of EcML with 50 mcg of Synsap 3 as the adjuvant, along with 0.3 mcg of H3N2.

### Example 1-19 to Example 1-23

The DOPC, cholesterol, TLR-4-1 and Synsap 3 were mixed and dissolved in methanol. Subsequently, the liposome was prepared according to the section of the preparation of liposomes as mentioned previously. A vaccine composition was prepared by combining a 10 mcg dose of TLR-4-1 with either 5 mcg, 50 mcg, 75 mcg or 100 mcg of Synsap 3 as the adjuvant, along with 0.3 mcg of H3N2. Another vaccine composition was prepared by combining a 5 mcg dose of TLR-4-1 with 50 mcg of Synsap 3 as the adjuvant, along with 0.3 mcg of H3N2.

### Example 1-24 to Example 1-25

The oil in water emulsion was prepared using the previously described in the preparation of oil in water emulsion method. A vaccine composition was prepared by combining a 5 mcg dose of CRX-527 (from InvivoGen.) with either 50 mcg or 100 mcg of Synsap 3 as the adjuvant, along with 0.3 mcg of H3N2.

### Example 1-26 to Example 1-30

The DOPC, cholesterol, TLR-4-1 and Synsap 1 were mixed and dissolved in methanol. Subsequently, the liposome was prepared according to the section of the preparation of liposomes as mentioned previously. A vaccine composition was prepared by combining a 10 mcg dose of TLR-4-1 (undisclosed TLR4-1) with either 5 mcg, 50 mcg, 75 mcg or 100 mcg of Synsap 1 as the adjuvant, along with 0.3 mcg of H3N2. Another vaccine composition was prepared by combining a 5 mcg dose of TLR-4-1 with 50 mcg of Synsap 1 as the adjuvant, along with 0.3 mcg of H3N2.

### Example 2: Preparation of the Vaccine Composition (Synsap/TLR2 Agonist)

### Example 2-1 to Example 2-3

A vaccine composition was prepared by combining a 10 mcg dose of Pam3CSK4 (from InvivoGen) with 1, 10 and 100 mcg of Synsap 5 as the adjuvant, TPGS (D-α-tocopheryl polyethylene glycol succinate) dissolved in propylene glycol and ethanol, followed by dilution with PBS solution. After that, 0.3 mcg of H3N2 was added to the mixture.

### Example 3: Preparation of the Vaccine Composition (Synsap/TLR7/8 Agonist)

### Example 3-1 to Example 3-4

A vaccine composition was prepared by combining a 10 mcg dose of R848 (from InvivoGen) with 2, 10, 50 and 100 mcg of Synsap 14 as the adjuvant, TPGS (D-α-tocopheryl polyethylene glycol succinate) dissolved in propylene glycol and ethanol, followed by dilution with PBS solution. After that, 0.3 mcg of H3N2 was added to the mixture.

### Example 4: Preparation of the Vaccine Composition (Synsap/TLR9 Agonist)

### Example 4-1 to Example 4-6

A vaccine composition was prepared by combining a 10 mcg dose of CpG 1018 (from InvivoGen) with either 10 mcg, 50 mcg, 75 mcg or 100 mcg of Synsap 17 as the adjuvant or 50 mcg dose of CpG 1018 with 10 mcg or 50 mcg of Synsap 17 as the adjuvant, along with 0.3 mcg of H3N2.

### Example 4-7 to Example 4-11

A vaccine composition was prepared by combining a 10 mcg dose of ODN 7909 (from InvivoGen) with either 10 mcg, 50 mcg, 75 mcg or 100 mcg of Synsap 3 as the adjuvant or 50 mcg dose of ODN 7909 with 10 mcg of Synsap 3 as the adjuvant, along with 0.3 mcg of H3N2.

### Example 5: Preparation of the Vaccine Composition (Synsap/TLR3 Agonist)

### Example 5-1 to Example 5-6

A vaccine composition was prepared by combining a 25 mcg dose of Poly(I:C) (from InvivoGen) with either 2.5, 5 mcg, 25 mcg, 50 mcg, 100 mcg or 250 mcg of Synsap 21 as the adjuvant, along with 0.3 mcg of H3N2.

### Example 5-7

A vaccine composition was prepared by combining a 25 mcg dose of TLR3-1 with 50 mcg of Synsap 21 as the adjuvant, along with 0.3 mcg of H3N2.

### Example a: Immunogenicity of Synsap Mixing with Different Ratios of TLR Agonist Candidates in B57CL/6

### Example A-1 to Example A-4

Mice were immunized intramuscularly (right leg quadriceps femoris muscle) with 0.3 mcg H3N2 alone, or with 5 mcg dose of TLR-4 agonist, 3D-PHAD in combination with either a 5 mcg, 50 mcg, 75 mcg and 100 mcg of Synsap 3 on Days 0 and 14. GSK's adjuvant AS01B was used as benchmark in this study. The vaccine formulations were prepared as mentioned in Example 1. Blood samples were collected at weeks 2 (Day 14), and 4 (Day 28) for monitoring of the H3N2 specific IgG antibody subclasses, HI titer. Additionally, at week 4, spleens were collected from mice to monitor the Ag-specific multifunctional CD4 T cell and the IL-4 and IFN-γ secretions.

### Results

Refer to Table 1. A 2-dose series of adjuvanted H3N2 vaccine, 2 out of 5 mice in the AS01B-adjuvanted H3N2 group were experienced more than a 10% body weight loss one day after each vaccination, but average body weight was less than 10%. However, the body weight of all vaccinated mice recovered within four days post-vaccination.

TABLE 1
Average body weight change (%)

| | Post-dose 1 | | | Post-dose 2 | | |
|---|---|---|---|---|---|---|
| Group | D 1 | D 2 | D 14 | D 15 | D 16 | D 21 |
| H3N2 | 0.515 | −0.064 | 6.111 | 3.153 | 5.322 | 4.979 |
| 1:1 3D-PHAD:Synsap 3 | −6.070 | −2.480 | 8.879 | −3.407 | 0.886 | 4.724 |
| 1:10 3D-PHAD:Synsap 3 | −7.569 | −8.302 | 6.718 | −6.946 | −5.785 | 0.851 |
| 1:15 3D-PHAD:Synsap 3 | −8.821 | −8.805 | 5.653 | −7.167 | −7.988 | −0.059 |
| 1:20 3D-PHAD:Synsap 3 | −6.071 | −6.199 | 6.660 | −6.668 | −8.891 | −0.985 |
| AS01B | −8.173 | −3.997 | 9.481 | −9.599 | −5.213 | 2.076 |
| 50 mcg Synsap 3 | 2.176 | 2.174 | 6.946 | −0.739 | 1.208 | 2.329 |

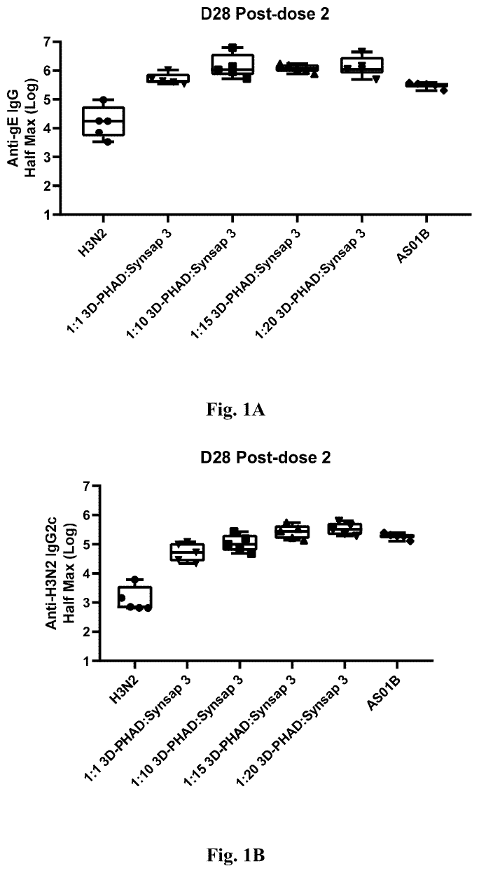

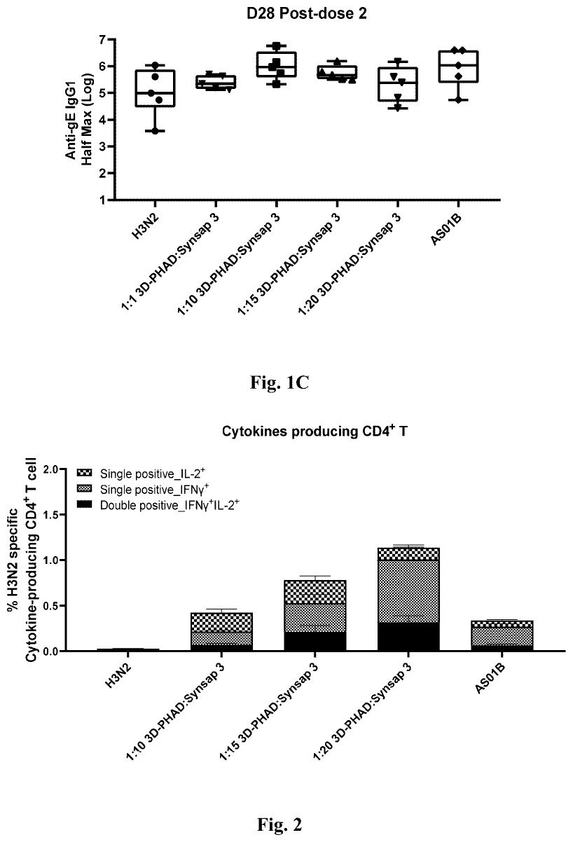

Anti-H3N2 IgG antibody titers and subtypes on 28 (post-dose 2) were presented in FIG. 1A. The adjuvanted H3N2 vaccines elicited total IgG titers approximately 1 to 2.5 logs higher than those observed in mice receiving unadjuvanted H3N2. The combination of 3D-PHAD and Synsap 3 exhibited no statistically significant difference in IgG induction compared to AS01B, but it showed a 4- to 6-fold increase comparing to antigen alone. Refer to FIG. 1B. The similar results were observed in IgG2c titers, indicating that SynSap induced a Th1-biased response in a dose-dependent manner. Refer to Table 2 below. Consistent with the anti-H3N2 antibody responses, higher HA neutralizing antibody responses after the boost (day 28) in all adjuvanted groups were observed compared to the control non-adjuvanted group, with the 3D-PHAD and SynSap 3 at the ratio 1:15 and 1:20, showing higher responses than AS01B.

TABLE 2
HI titer and comparisons.

| | GMT | Fold (compare | Fold (compare |
|---|---|---|---|
| Group | mean | to H3N2) | to AS01B) |
| H3N2 | 160 | 1.0 | 0.125 |
| 1:1 3D-PHAD:Synsap 3 | 970 | 6.1 | 0.8 |
| 1:10 3D-PHAD:Synsap 3 | 1280 | 8 | 1 |
| 1:15 3D-PHAD:Synsap 3 | 3378 | 21 | 2.6 |
| 1:20 3D-PHAD:Synsap 3 | 1689 | 10 | 1.3 |
| AS01B | 1280 | 8 | 1 |

To clarify the cellular immune response induced by vaccination, groups including H3N2 alone, AS01B, 1:10, 1:15 and 1:20 of 3D-PHAD and SynSap 3 were included. Splenocytes were isolated and re-stimulated in vitro with H3N2 antigen, followed by intracellular staining to detect IFNγ and IL-2 production. Analysis of H3N2-specific CD4⁺ T cell subsets in immunized mice was measured by flow cytometry (FIG. 2). The non-adjuvanted H3N2 groups did not induce noticeable cytokine production in CD4 T cells. In contrast, the 1:15 and 1:20 of 3D-PHAD and Synsap 3 induced significant cytokine production by total CD4⁺ T cells at 2 weeks post-boost compared to AS01B group, with the p values of 0.0016 and <0.0001, respectively. Additionally, in all mice, those adjuvanted with 1:10 3D-PHAD and Synsap 3 showed a trend towards higher cytokine levels compared to AS01B group, although not statistically significant. Further, the 1:10, 1:15 and 1:20 of 3D-PHAD and Synsap 3 induced significant cytokine production by total CD4⁺ T cells at 2 weeks post-boost compared to H3N2, with the p values of 0.0101, <0.0001, and <0.0001, respectively.

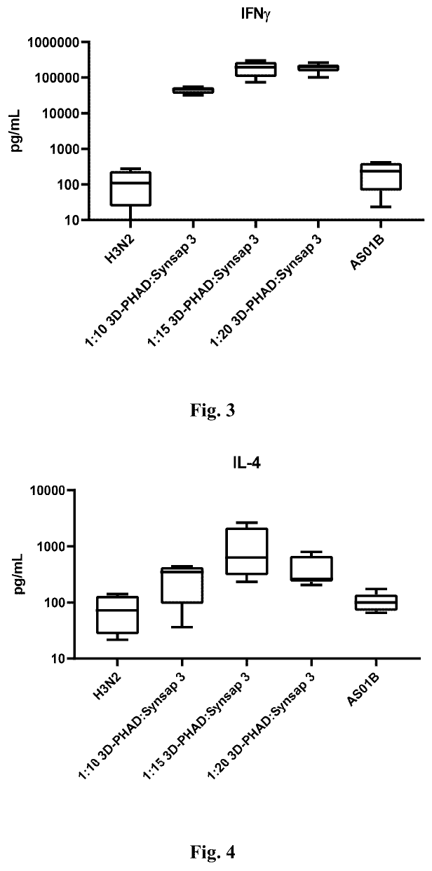

Antigen-specific cytokine production was assessed in splenocytes. Secreted cytokines were measured via ELISA assay after H3N2 stimulation (FIG. 3 and FIG. 4.). The combination of 3D-PHAD and Synsap 3 groups exhibited dramatic increased IFNγ production compared to the H3N2 and AS01B groups, but less detectable IL-4 responses, align with Synsap 3's Th1-biased profile. The inclusion of Synsap 3 in the TLR4 formulation appeared to modulate the immune response, potentially by triggering both Th1 pathways, as evidenced by enhanced IFNγ secretion.

### Example A-5 to Example A-10

Mice were immunized intramuscularly (right quadriceps femoris muscle) with 0.3 mcg of H3N2 alone or with 50 mcg of Synsap 3 or Synsap 1 in combination with 5 mcg dose of a series of TLR4 agonists (comprising PHAD, 3D-PHAD, 3D-6A-PHAD, CRX-527, EcML, and TLR4-1 on Days 0 and 14. PHAD, 3D-PHAD, 3D-6A-PHAD were obtained from Avanti. CRX-527 was obtained from InvivoGen. EcML was obtained from EuBiologics. GSK's adjuvant AS01B was as benchmark in this study. The vaccine formulations were prepared as mentioned in Example 1. Blood samples were collected at weeks 2 and 4 for monitoring of the H3N2 specific IgG antibody subclasses, HI titer. Additionally, at weeks 4, spleens were collected from mice to monitor the Ag-specific multifunctional CD4⁺ T cell and the IL-4 and IFN-g secretions.

### Results

The ability of the combination of Synsap 3 and other TLR4 agonists adjuvanted H3N2 to induce humoral and cellular immune response was evaluated in C57BL/6 mice. Refer to Table 3. During the study, mice in the AS01B and TLR4/Synsap groups exhibited less than or approximately 10% average weight changes one day post-vaccination. However, the body weight of all vaccinated mice recovered within four days post-vaccination.

TABLE 3
Average body weight change (%)

| | Post-dose 1 | | | Post-dose 2 | | |
|---|---|---|---|---|---|---|
| Group | D 1 | D 2 | D 14 | D 15 | D 16 | D 21 |
| 0.3 mcg H3N2 | 0.515 | −0.064 | 6.272 | 3.153 | 5.322 | 5.513 |
| 5 mcg 3D-6A-PHAD | −5.362 | −2.900 | 6.215 | −1.147 | 0.463 | 2.331 |
| 5 mcg EcML | −3.607 | −1.832 | 7.914 | −3.002 | −0.735 | 1.468 |
| 10 mcg TLR4-1 | −8.384 | −4.650 | 4.990 | −7.086 | −8.750 | −1.984 |
| 5/50 mcg TLR4-1/Synsap 1 | −3.290 | −2.100 | 7.246 | −3.9565 | −3.8723 | 0.260 |
| 5/50 mcg TLR4-1/Synsap 3 | −5.653 | −3.804 | 8.772 | −9.683 | −9.694 | −6.052 |
| 5/50 mcg EcML/Synsap 1 | −4.860 | −3.643 | 8.756 | −8.599 | −10.324 | −3.715 |
| 5/50 mcg CRX-527/Synsap 3 | −4.126 | 0.447 | 10.867 | −6.815 | −4.991 | −0.584 |
| 5/50 mcg PHAD/Synsap 3 | −5.441 | −2.717 | 7.820 | −9.229 | −9.827 | −0.541 |
| 5/50 mcg 3D-PHAD/Synsap 3 | 0.009 | 3.000 | 8.775 | −7.397 | −7.114 | 0.946 |
| 5/50 mcg 3D-6A-PHAD/Synsap 3 | −3.217 | −0.085 | 7.709 | −9.626 | −9.315 | −1.644 |
| 5/5 mcg AS01B | −8.173 | −3.997 | 9.005 | −9.599 | −5.213 | 1.550 |
| 50 mcg Synsap 1 | −1.096 | −1.736 | 3.753 | 2.417 | −1.393 | 6.148 |
| 50 mcg Synsap 3 | 2.176 | 2.174 | 6.946 | −0.739 | 1.208 | 2.329 |

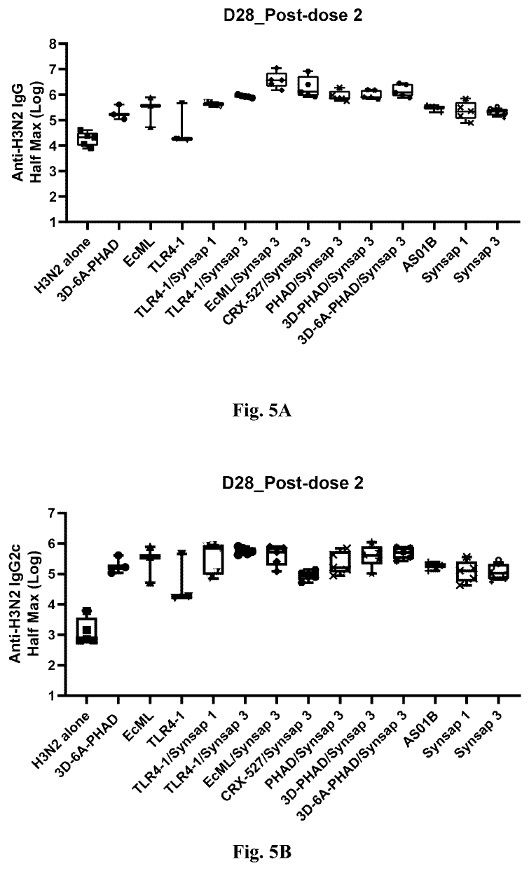

Antigen-specific antibody titers were measured by ELISA. The anti-H3N2 antibody titer was measured on Day 28 and is expressed as the dilution of serum exhibiting half-maximal binding (optical density: 50%) based on its absorbance at 450 nm. The total IgG (FIG. 5A), IgG2c (FIG. 5B), and IgG1 (FIG. 5C) at day 28 were measured in immunized mice. IgG2c and IgG1 titers are associated with Th1 and Th2 responses, respectively. Refer to FIG. 5A. On Days 28 post-immunization, anti-H3N2 IgG antibody titers and subtypes were analyzed. The adjuvanted H3N2 vaccines demonstrated total IgG titers approximately 1 to 2.5 logs higher than those observed in unadjuvanted H3N2 mice. Moreover, adjuvants the combination-based and AS01B enhanced IgG titers when compared with TLR4 agonist alone and SynSap 3 alone adjuvanted groups. Refer to FIG. 5B. After the booster dose, the TLR4-1, 3D-PHAD, 3D-6A-PHAD and EcML combined with Synsap 3 groups demonstrated statistically significant differences in anti-H3N2 IgG2c titers when compared to AS01B among the adjuvanted groups, and all adjuvanted groups showed significantly higher titers compared to the unadjuvanted group. These results containing the combination-based adjuvants exhibited a higher antibody response and elicit Th1 biased response following the immunizations. As shown in Table 4, consistent with the anti-H3N2 antibody responses, all adjuvanted groups exhibited higher HA neutralizing antibody responses after the boost (day 28) compared to the non-adjuvanted control group. Notably, the TLR4-1/Synsap 3 and 3D-6A-PHAD/Synsap 3 groups demonstrated at least a twofold increase in titer compared to AS01B.

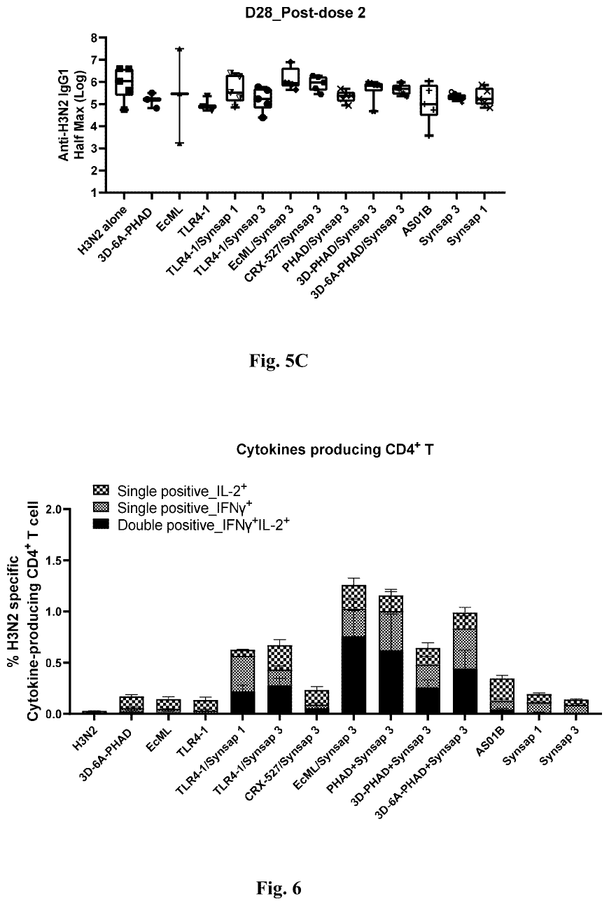

To evaluate the cellular immune response induced by vaccination, splenocytes were isolated 14 days after the booster dose and restimulated in vitro with the H3N2 antigen. Intracellular staining was then performed to detect IFNγ and IL-2 production, enabling the assessment of multifunctional CD4 T cells. Analysis of H3N2-specific CD4⁺ T cell subsets in immunized mice were measured by flow cytometry. Refer to FIG. 6. Mice immunized with Synsap 3 (50 mcg) in combination with TLR4 agonists-EcML (5 mcg), PHAD (5 mcg), and 3D-6A-PHAD (5 mcg) showed significantly increased multiple cytokine production by CD4 T cells compared to groups immunized with AS01B, with the p values of 0.0002, 0.009, and 0.0081, respectively. Mice immunized with Synsap in combination with TLR4 agonists-EcML, PHAD, 3D-PHAD, 3D-6A-PHAD, and TLR4-1, and Synsap 1 (50 mcg) in combination with TLR4-1 showed also significantly increased multiple cytokine production by CD4 T cells compared to groups immunized with H3N2 alone, with the p values of <0.0001, <0.0001, 0.0115, <0.0001, 0.084, and 00139, respectively.

TABLE 4
HI titers and comparisons

| | GMT | Fold (compare | Fold (compare |
|---|---|---|---|
| Group | mean | to H3N2) | to AS01B) |
| H3N2 | 160 | 1.0 | 0.1 |
| 3D-6A-PHAD | 806.3 | 5.0 | 0.6 |
| TLR4-1 | 403.2 | 2.5 | 0.3 |
| EcML | 403.2 | 2.5 | 0.3 |
| TLR4-1/Synsap 1 | 2229 | 14 | 1.7 |
| TLR4-1/Synsap 3 | 3378 | 21 | 2.6 |
| CRX-527/Synsap 3 | 1280 | 8 | 1 |
| PHAD/Synsap 3 | 1280 | 8 | 1 |
| 3D-PHAD/Synsap 3 | 1470 | 9.2 | 1.1 |
| 3D-6A-PHAD/Synsap 3 | 2560 | 16 | 2 |
| EcML/Synsap 3 | 1689 | 10.6 | 1.3 |
| AS01B | 1280 | 8 | 1 |
| Synsap 1 | 320 | 2 | 0.25 |
| Synsap 3 | 192 | 1.2 | 0.15 |

### Example B: Immunogenicity of Synsap 5+TLR2 Agonist in B57CL/6

Mice were immunized intramuscularly (right quadriceps femoris muscle) with 0.3 mcg of H3N2 alone or with 100 mcg of Synsap 5 in combination with a 10 mcg dose of a TLR2 agonist, Pam3CSK4, on Days 0 and 14. The Pam3CSK4 was obtained from InvivoGen. The vaccine formulations were prepared as mentioned in Example 2. Blood samples were collected at weeks 2, and 4 for monitoring of the H3N2 specific IgG antibody subclasses, HI titer.

### Results

The ability of the combination of Synsap 5 and TLR2 agonist adjuvanted H3N2 to induce humoral immune response was evaluated in C57BL/6 mice. Refer to Table 5. During the experiment, no serious body weight loss, which means loss over 10%, was observed after vaccination.

TABLE 5
Body weight change (%)

| | Post-dose 1 | | | Post-dose 2 | | |
|---|---|---|---|---|---|---|
| Group | D 1 | D 2 | D 14 | D 15 | D 16 | D 21 |
| H3N2 | 0.515 | −0.064 | 6.272 | 3.153 | 5.322 | 5.513 |
| Pam3CSK4 | −0.996 | 2.635 | 6.308 | −0.998 | 0.169 | 4.551 |
| 10:1 Synsap | −4.402 | 0.799 | 4.817 | −0.500 | 2.257 | 7.314 |
| 5:Pam3CSK4 | | | | | | |
| Synsap 5 | −3.166 | 1.523 | 4.909 | −0.174 | 3.144 | 7.000 |

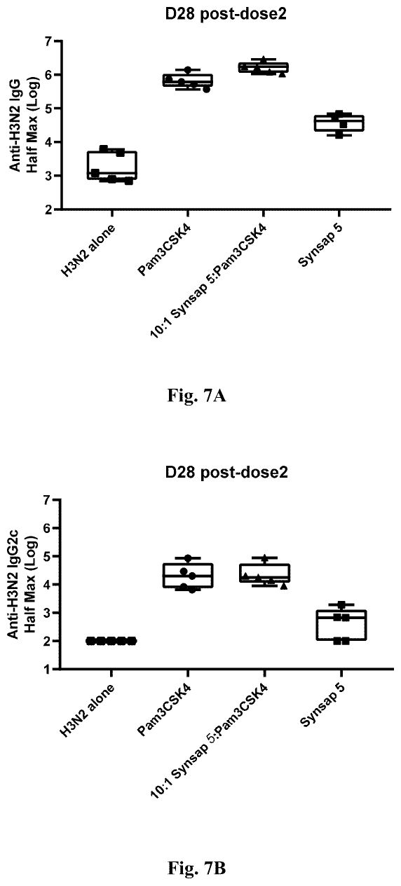

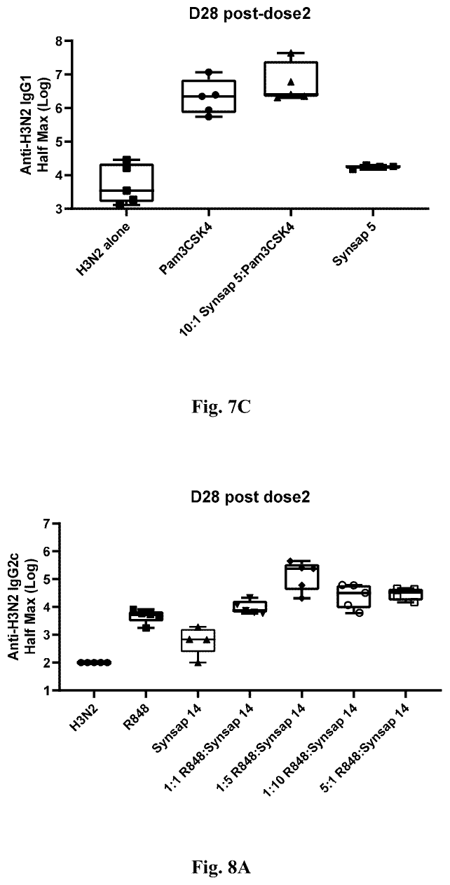

Antigen-specific antibody titers measured by ELISA. The anti-H3N2 antibody titer was measured on Day 28 and is expressed as the dilution of serum exhibiting half-maximal binding (optical density: 50%) based on its absorbance at 450 nm. The total IgG, IgG2c, and IgG1 at day 28 were measured in immunized mice. IgG2c and IgG1 titers are associated with Th1 and Th2 responses, respectively. On day 14 (post-dose 1) and 28 (post-dose 2) anti-H3N2 IgG antibody titers and subtypes are presented in FIG. 7A to FIG. 7C. The adjuvanted H3N2 vaccines significantly elicited total IgG titers production than those observed in mice receiving unadjuvanted H3N2. The combination of Synsap 5 and TLR2 agonist significantly increased total IgG by 2 logs over the unadjuvanted control, and show differences between the Synsap 5, and Pam3CSK4 groups. Refer to Table 6, consistent with the anti-H3N2 antibody responses, higher hemagglutination inhibition (HI) neutralizing antibody responses were observed after the boost (day 28) in all combination-adjuvanted groups compared to the unadjuvanted control. Overall, the combination of Pam3CSK4 o with Synsap 5 demonstrates a synergistic effect on humoral immunity.

TABLE 6
HI Titers and Comparisons

| | | Fold | Fold | Fold |
|---|---|---|---|---|
| | GMT | (compare | (compare | (compare |
| Group | mean | to H3N2) | to CpG1018) | to SynSap 17) |
| H3N2 | 160 | 1.0 | 0.1 | 0.1 |
| Pam3CSK4 | 2176 | 14 | 1.0 | 1.1 |
| 10:1 | 2560 | 16.0 | 1.2 | 1.3 |
| Synsap 5:Pam3CSK4 | | | | |
| Synsap 5 | 2048 | 13 | 0.9 | 1.0 |

### Example C: Immunogenicity of Synsap 14+TLR7/8 Agonist in B57CL/6

Mice were immunized intramuscularly (right quadriceps femoris muscle) with 0.3 mcg of H3N2 alone or with 50 mcg of Synsap 14 in combination with a 10 mcg dose of a TLR7/8 agonist (Resiquimod, R848) on Days 0 and 14. R848 was obtained from InvivoGen. The vaccine formulations were prepared as mentioned in Example 3. Blood samples were collected at weeks 2, and 4 for monitoring of the H3N2 specific IgG antibody subclasses, HI titer. Additionally, at week 4, spleens were collected from mice to monitor the Ag-specific multifunctional CD4 T cell and the IL-4 and IFNγ secretions.

### Results

Refer to Table 7. During the experiment, no serious body weight loss (over 10%) was observed after vaccination. The ability of the combination of Synsap 14 and R848 adjuvanted H3N2 to induce humoral and cellular immune response was evaluated in C57BL/6 mice.

TABLE 7
Body weight change (%)

| | Post-dose 1 | | | Post-dose 2 | | |
|---|---|---|---|---|---|---|
| Group | D 1 | D 2 | D 14 | D 15 | D 16 | D 21 |
| H3N2 | −1.153 | 1.480 | 7.352 | −1.900 | −1.542 | −1.481 |
| R848 | −3.012 | −0.416 | 5.224 | −4.078 | −0.804 | 1.856 |
| Synsap 14 | −1.769 | −0.704 | 3.586 | −0.277 | −0.654 | 3.064 |
| 1:1 R848/Synsap 14 | −2.079 | 1.645 | 8.520 | −3.125 | 0.812 | 1.701 |
| 1:5 R848/Synsap 14 | −2.019 | 1.362 | 10.692 | −2.733 | −0.206 | 0.173 |
| 1:10 R848/Synsap 14 | −2.044 | 0.606 | 8.691 | −3.368 | −0.938 | 2.902 |
| 5:1 R848/Synsap 14 | −4.941 | −2.019 | 7.677 | −4.567 | −1.218 | 0.631 |

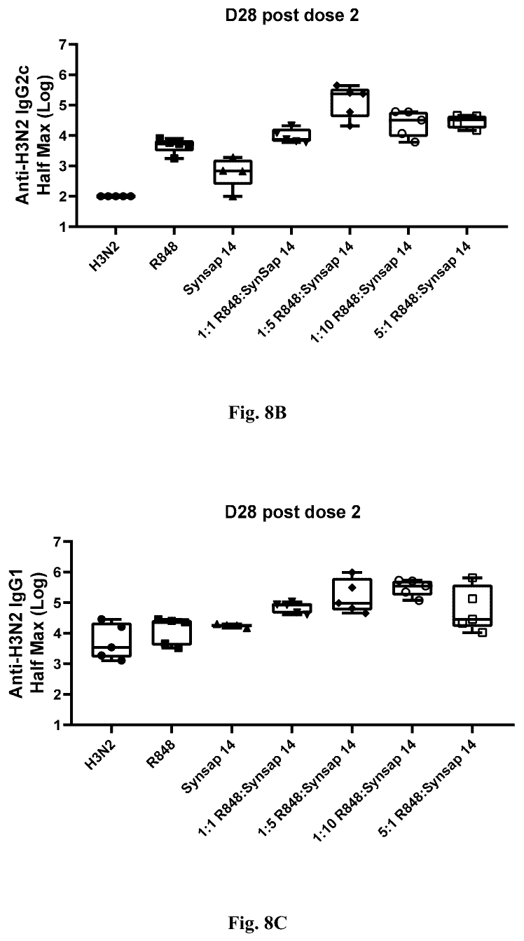

Antigen-specific antibody titers were measured by ELISA. The anti-H3N2 antibody titer was measured on Day 28 and is expressed as the dilution of serum exhibiting half-maximal binding (optical density: 50%) based on its absorbance at 450 nm. The total IgG, IgG2c, and IgG1 at day 28 (B) were measured in immunized mice. IgG2c and IgG1 titers are associated with Th1 and Th2 responses, respectively. Refer to FIG. 8A to FIG. 8C. On Days 28 post-immunization, anti-H3N2 IgG antibody titers and subtypes were analyzed. The adjuvanted H3N2 vaccines demonstrated total IgG titers approximately 0.5 to 2 logs higher than those observed in unadjuvanted H3N2 mice. Moreover, in all mice, those adjuvanted with the combination of Synsap 14 and R848 enhanced IgG titers when compared with R848 alone, and the ratio at 1:5 of Synsap 14 and R848 adjuvanted group demonstrated statistically significant differences in anti-H3N2 IgG and IgG2c titers, with the p value of <0.0001. These results containing the combination-based adjuvants exhibited a higher antibody response and elicit Th1 biased response following the immunizations. Consistent with the anti-H3N2 antibody responses, all adjuvanted groups exhibited higher HA neutralizing antibody responses after the boost (day 28) compared to the non-adjuvanted control group (Table 8). Notably, the all of the combination of Synsap 14 and R848 groups demonstrated at least a 1.5-fold increase in titer compared to R848 alone, or Synsap 14 alone.

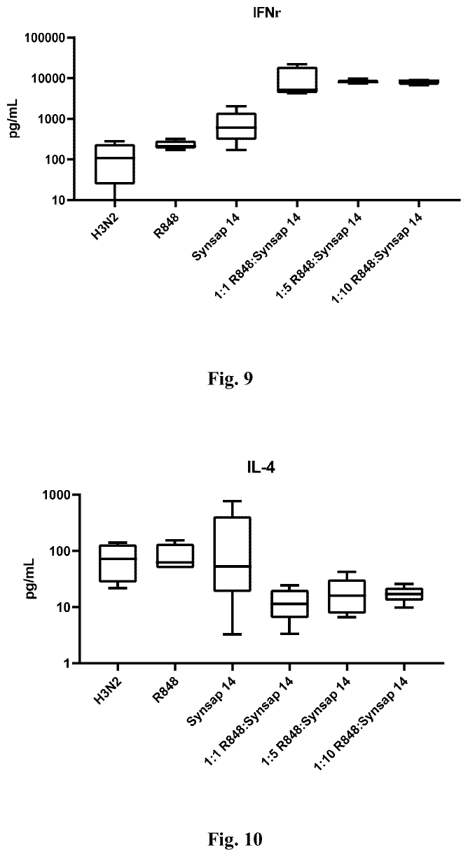

To evaluate the cellular immune response induced by vaccination, splenocytes were isolated 14 days after the booster dose and restimulated in vitro with the H3N2 antigen. Intracellular staining was then performed to detect IFNγ and IL-2 production, enabling the assessment of multifunctional CD4 T cells. Secreted cytokines were measured via ELISA assay after H3N2 stimulation. Refer to FIG. 9A to FIG. 9B. Mice immunized with Synsap 14 in combination with R848 showed significantly increased multiple cytokine production by CD4 T cells compared to groups immunized with AS01B and H3N2 alone. To further characterize the Th1/Th2/Th17 responses, antigen-specific cytokine production was assessed in splenocytes. Secreted cytokines were measured via ELISA assay after H3N2 stimulation. Refer to FIG. 9 to FIG. 11, H3N2 groups adjuvanted with the combination of R848 and Synsap 14 exhibited a trend towards increased IFNγ and IL-17 A production compared to the Synsap 14, and R848 groups. This suggests that the combination enhances immune activation, promoting a shift toward a Th1/Th17 immune response, which might be good for bacterial/protozoa vaccines. Overall, the combination of R848 with Synsap14 demonstrates a synergistic effect on humoral and cellular immunity.

TABLE 8
HI titer and comparsions

| | | | Fold | Fold |
|---|---|---|---|---|
| | | Fold | (compare | (compare |
| | | (compare | to R848 | to SynSap 14 |
| | GMT | to H3N2 | treated | treated |
| Group | mean | alone) | group) | group) |
| H3N2 | 160 | 1.0 | 0.9 | 0.8 |
| R848 | 183.8 | 1.1 | 1.0 | 1.0 |
| SynSap 14 | 192 | 1.2 | 1.0 | 1.0 |
| 1:1 R848/Synsap 14 | 278.6 | 1.7 | 1.5 | 1.5 |
| 1:5 R848/Synsap 14 | 735.2 | 4.6 | 4.0 | 3.8 |
| 1:10 R848/Synsap 14 | 367.6 | 2.3 | 2.0 | 1.9 |
| 5:1 R848/Synsap 14 | 320 | 2.0 | 1.7 | 1.7 |

### Example D: Immunogenicity of Synsap 17+TLR9 Candidates in B57CL/6

Mice were immunized intramuscularly (right quadriceps femoris muscle) with 0.3 mcg of H3N2 alone or with 10 mcg dose of a TLR 9 agonist, CpG 1018, in combination with either a 10 mcg, 50 mcg, 75 mcg and 100 mcg of Synsap 17, or 50 mcg CpG 1018 with 10 mcg Synsap 17 on Days 0 and 14. CpG1018 was obtained from InvivoGen. The vaccine formulations were prepared as mentioned in Example 4. Blood samples were collected at weeks 2, and 4 for monitoring of the H3N2 specific IgG antibody subclasses, HI titer. Additionally, at weeks 4, spleens were collected from mice to monitor the Ag-specific multifunctional CD4 T cell and the IL-4 and IFN-g secretions.

### Results

The ability of the combination of Synsap 17 and TLR9 agonist adjuvanted H3N2 to induce humoral and cellular immune response was evaluated in C57BL/6 mice. Refer to Table 9. No mouse exhibited a weight loss exceeding 10% following vaccination.

TABLE 9
Body weight change (%)

| | Post-dose 1 | | | Post-dose 2 | | |
|---|---|---|---|---|---|---|
| Group | D 1 | D 2 | D 14 | D 15 | D 16 | D 21 |
| H3N2 | −1.153 | 1.480 | 7.352 | −1.900 | −1.542 | −0.820 |
| CpG 1018 | −5.463 | −7.285 | 4.568 | −2.546 | −7.323 | 1.850 |
| Synsap 17 | −1.769 | −0.704 | 3.586 | −0.277 | −0.654 | 2.921 |
| 1:1 | −3.500 | −6.986 | 0.255 | 0.944 | −3.841 | 3.191 |
| CpG 1018:Synsap 17 | | | | | | |
| 1:5 | −0.385 | −1.953 | 3.447 | −0.254 | −1.145 | 5.159 |
| CpG 1018:Synsap 17 | | | | | | |
| 1:7.5 | −1.765 | −1.808 | 3.327 | −0.201 | −0.719 | 6.178 |
| CpG 1018:Synsap 17 | | | | | | |
| 1:10 | −4.675 | −2.366 | 1.477 | −1.193 | −1.716 | 4.577 |
| CpG 1018:Synsap 17 | | | | | | |
| 5:1 | −7.035 | −7.286 | 1.598 | −1.900 | −6.982 | 2.319 |
| CpG 1018:Synsap 17 | | | | | | |

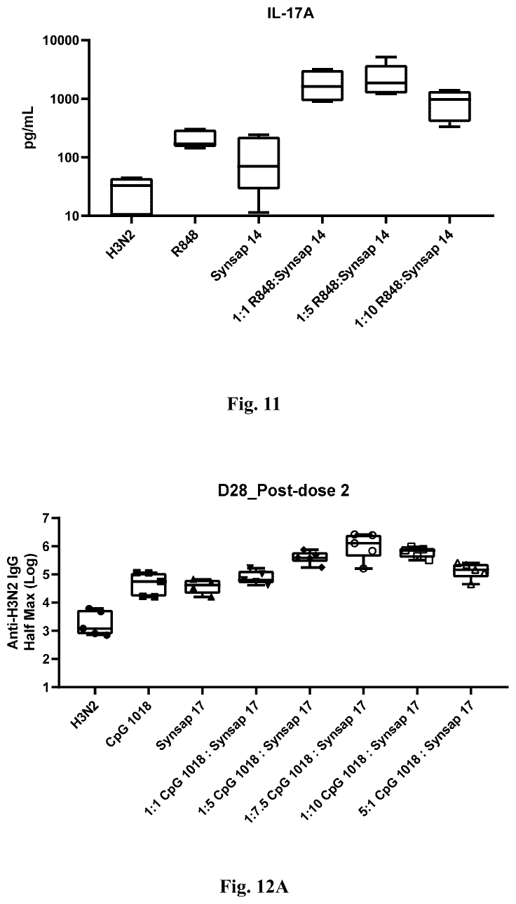

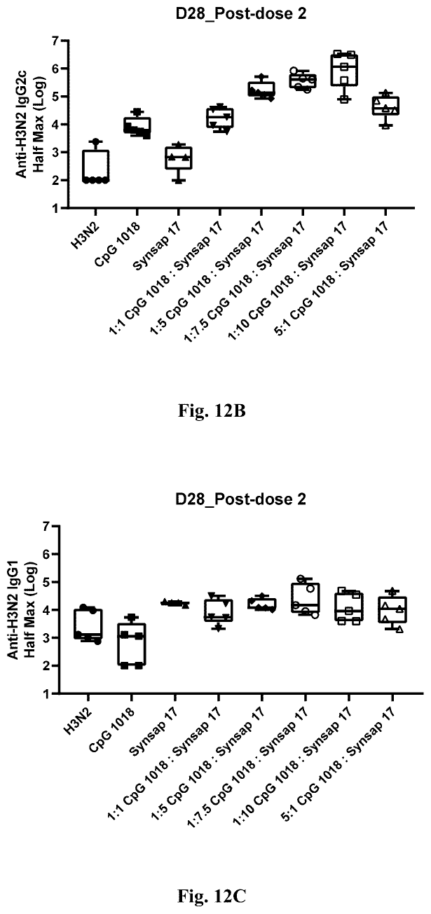

Antigen-specific antibody titers were measured by ELISA. The anti-H3N2 antibody titer was measured on Day 28 and was expressed as the dilution of serum exhibiting half-maximal binding (optical density: 50%) based on its absorbance at 450 nm. The total IgG, IgG2c, and IgG1 at day 28 were measured in immunized mice. IgG2c and IgG1 titers are associated with Th1 and Th2 responses, respectively. FIG. 12A to FIG. 12C present anti-H3N2 IgG antibody titers and subtypes on days 14 and 28 (post-dose 1 and 2) for each group. After the first dose, H3N2 vaccines adjuvanted with the combination of Synsap 17 and TLR9 agonist elicited total IgG titers approximately 0.5 to 1.5 logs higher than those observed in unadjuvanted, Synsap 17 and CpG 1018 groups. Increasing the dose of Synsap 17 in the combination could enhance the total IgG titer and IgG2c. Consistent with the anti-H3N2 antibody responses, higher hemagglutination inhibition neutralizing antibody responses were observed after the boost (day 28) in all combination-adjuvanted groups compared to the unadjuvanted control, with the 1:10 Synsap 17 and CpG 1018 group demonstrating significantly higher responses (Table 10).

TABLE 10
HI titer and comparisions

| | | Fold | Fold | Fold |
|---|---|---|---|---|
| | GMT | (compare | (compare to | (compare to |
| Group | mean | to H3N2) | CpG1018) | Synsap 17) |
| H3N2 | 160 | 1.0 | 0.5 | 0.9 |
| CpG 1018 | 320 | 2.0 | 1.0 | 1.7 |
| Synsap 17 | 183.8 | 1.1 | 0.6 | 1.0 |
| 1:1 CpG | 367.6 | 2.3 | 1.1 | 2.0 |
| 1018:Synsap 17 | | | | |
| 1:5 CpG | 970.1 | 6.1 | 3.0 | 5.3 |
| 1018:Synsap 17 | | | | |
| 1:7.5 | 1114 | 7.0 | 3.5 | 6.1 |
| CpG 1018:Synsap 17 | | | | |
| 1:10 | 2560 | 16 | 8.0 | 14 |
| CpG 1018:Synsap 17 | | | | |
| 5:1 CpG | 640 | 4.0 | 2.0 | 3.5 |
| 1018:Synsap 17 | | | | |

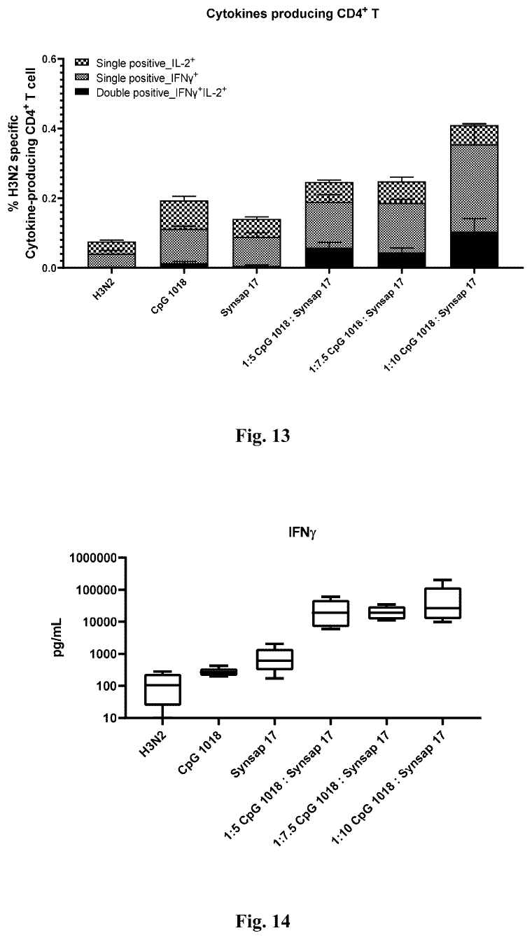

To investigate the cellular immune response, splenocytes were isolated from mice immunized with H3N2 alone, 1:5, 1:7.5 and 1:10 of the combination of CpG 1018 and Synsap 17, Synsap 17 and CpG 1018. Splenocytes were re-stimulated with H3N2 antigen, followed by intracellular cytokine staining to assess IFNγ and IL-2 production (FIG. 13). While the non-adjuvanted H3N2 group did not induce significant cytokine production in CD4 T cells, the 1:5, 1:7.5 and 1:10 of the combinations of Synsap 17 and CpG 1018 elicited significantly higher levels of cytokine production by total CD4 T cells compared to un-adjuvanted, Synsap 17 and CpG 1018 groups. Specifically, the 1:5, 1:7.5, and 1:10 of the combinations of Synsap 17 and CpG 1018 showed significantly increased cytokine production compared to H3N2 with the p values of 0.0001, 0.0001, and <0.0001, respectively.

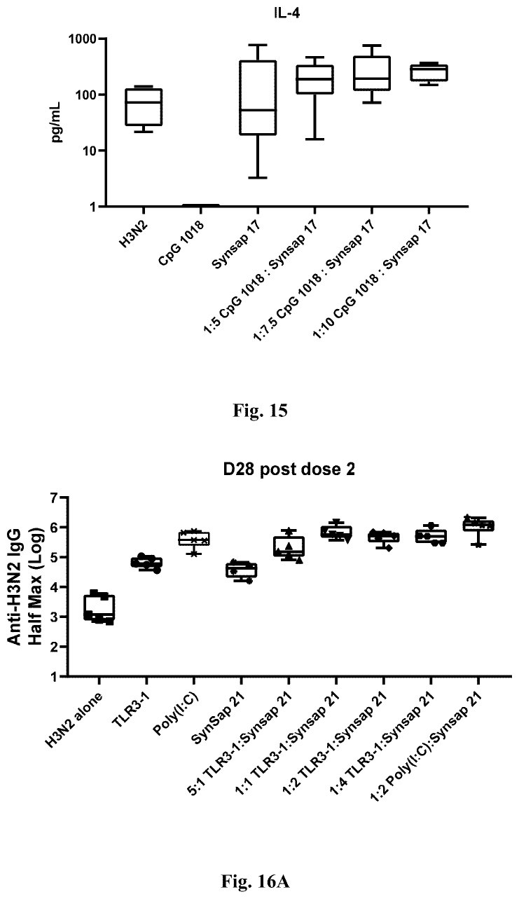

To further characterize the Th1/Th2 responses, antigen-specific cytokine production was assessed in splenocytes. Secreted cytokines were measured via ELISA assay after H3N2 stimulation. Refer to FIG. 14 to FIG. 15. H3N2 groups adjuvanted with the combination of CpG 1018 and Synsap 17 exhibited a trend towards increased IFN-γ production compared to the Synsap 17 and CpG 1018 groups. CpG-adjuvanted groups lacked detectable IL-4 responses, align with a Th1-biased profile. The inclusion of Synsap 17 in the CpG formulation appeared to modulate the immune response, potentially by triggering both Th1 and Th2 pathways, as evidenced by enhanced IFN-γ and IL-4 secretion.

### Example E: Immunogenicity of Synsap 21+TLR3 Agonists in B57CL/6

Mice were immunized intramuscularly (right quadriceps femoris muscle) with 0.3 mcg of H3N2 alone or with 5 mcg, 25 mcg, 50 mcg and 100 mcg of Synsap 21 in combination with a 25 mcg dose of a TLR3 agonist comprising Poly (I:C) and TLR3-1 on Days 0 and 14. Poly (I:C) was obtained from InvivoGen, and TLR3-1 is an undisclosed TLR3 compound. The vaccine formulations were prepared as mentioned in Example 5. Blood samples were collected at weeks 2, and 4 for monitoring of the H3N2 specific IgG antibody subclasses, HI titer. Additionally, at week 4, spleens were collected from mice to monitor the Ag-specific multifunctional CD4 T cell and the IL-4 and IFN-g secretions.

### Results

The ability of the combination of Synsap 21 and TLR3 agonist adjuvanted H3N2 to induce humoral and cellular immune response was evaluated in C57BL/6 mice. Refer to Table 11. During the experiment, no serious body weight loss (over 10%) was observed after vaccination.

TABLE 11
Average body weight loss

| | Post-dose 1 | | | Post-dose 2 | | |
|---|---|---|---|---|---|---|
| Group | D 1 | D 2 | D 14 | D 15 | D 16 | D 21 |
| H3N2 alone | −1.153 | 1.480 | 7.565 | −1.900 | −1.542 | −0.820 |
| TLR3-1 | −4.647 | 0.769 | 6.060 | −5.203 | −2.131 | −0.204 |
| 1:5 Synsap 21:TLR3-1 | −4.527 | 1.505 | 7.444 | −1.900 | −1.542 | −0.820 |
| 1:1 Synsap 21:TLR3-1 | −4.764 | −1.090 | 8.265 | −6.418 | −1.209 | 2.807 |
| 2:1 Synsap 21:TLR3-1 | −5.886 | −1.211 | 10.902 | −8.842 | −4.266 | 0.496 |
| 4:1 Synsap 21:TLR3-1 | −6.864 | −3.310 | 9.327 | −8.637 | −6.051 | 0.185 |
| Poly(I:C) | −4.287 | 1.634 | 9.773 | −5.203 | −2.131 | −0.204 |
| 2:1 Synsap 21:Poly(I:C) | −4.724 | −0.453 | 8.007 | −5.150 | −1.825 | 0.022 |
| SynSap 21 | 2.176 | 2.174 | 6.946 | −0.739 | 1.208 | 0.149 |

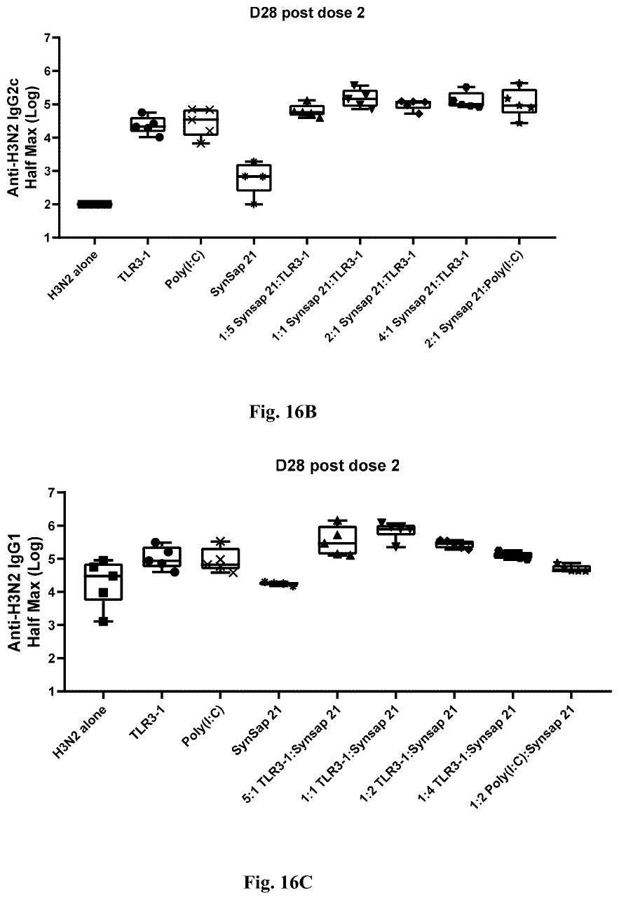

Antigen-specific antibody titers were measured by ELISA. The anti-H3N2 antibody titer was measured on Day 28 and was expressed as the dilution of serum exhibiting half-maximal binding (optical density: 50%) based on its absorbance at 450 nm. The total IgG, IgG2c, and IgG1 at day 28 were measured in immunized mice. IgG2c and IgG1 titers are associated with Th1 and Th2 responses, respectively. On day 28 (post-dose 2) anti-H3N2 IgG antibody titers and subtypes are presented in FIG. 16A. The adjuvanted H3N2 vaccines significantly elicited total IgG titers production than those observed in mice receiving unadjuvanted H3N2. No significant differences in results were found between these two different TLR3 agonists. Total IgG and IgG2c were significantly increased by 1.5 to 2 logs by the combination of Synsap 21 and TLR3 agonists at all doses over the unadjuvanted control, showing differences between the Synsap 21, TLR3-1 and Poly (I:C) groups (FIGS. 16B and 16C). Consistent with the anti-H3N2 antibody responses, higher hemagglutination inhibition neutralizing antibody responses were observed after the boost (day 28) in all combination-adjuvanted groups compared to the unadjuvanted control (Table 12). Overall, a synergistic effect on IgG and IgG2c titers and HI titer generated by the combination of TLR3-1 or Poly(I:C) with Synsap 21 was demonstrated. In addition, Th1 responses tends to be promoted by the combination of TLR3-1 or Poly(I:C) with Synsap 21.

TABLE 12
HI titer and comparsions

| | | Fold | Fold | Fold |
|---|---|---|---|---|
| | GMT | (compare | (compare | (compare to |
| Group | mean | to H3N2) | to TLR3) | SynSap 21) |
| H3N2 | 160 | 3.03125 | 1 | 1.515625 |
| TLR3-1 | 485 | 2 | 0.659794 | 1 |
| 1:5 Synsap 21:TLR3-1 | 557.2 | 5.278125 | 1.741237 | 2.639063 |
| 1:1 Synsap 21:TLR3-1 | 844.5 | 6.063125 | 2.000206 | 3.031563 |
| 2:1 Synsap 21:TLR3-1 | 970.1 | 8 | 2.639175 | 4 |
| 4:1 Synsap 21:TLR3-1 | 1280 | 4.595 | 1.14875 | 2.2975 |
| Poly(I:C) | 640 | 1 | 0.329897 | 0.5 |
| 2:1 Synsap 21:Poly(I:C) | 735.2 | 4 | 1 | 2 |
| Synsap 21 | 320 | 3.4825 | 1.148866 | 1.74125 |

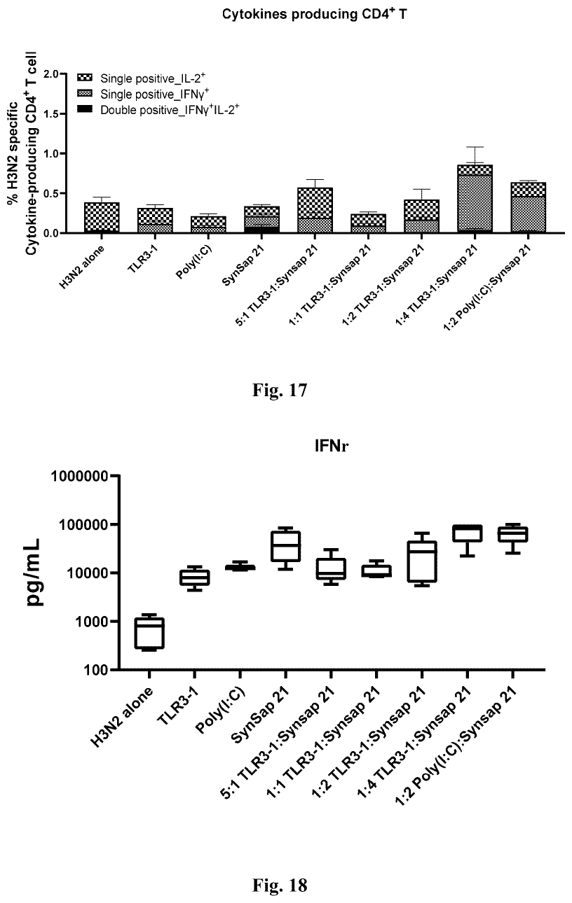

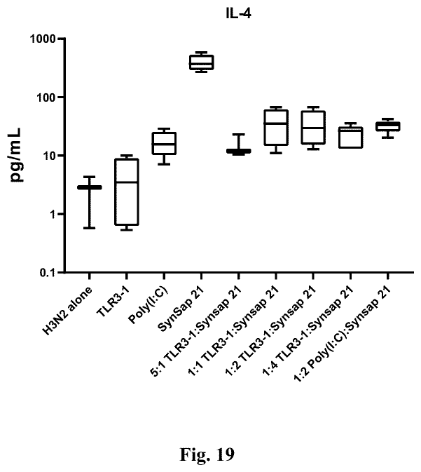

The data in FIG. 17 clearly indicates that all tested adjuvants activate polyfunctional CD4⁺ and H3N2-specific IFNγ secretion. Specifically, the combinations of TLR3-1 and Synsap 21 with the ratio of 2:1 and 4:1 respectively, and the combination of Poly(I:C) and Synsap 21 with the ratio of 2:1 induced significant populations in splenic CD4⁺ Th1 cells and antigen-specific IFNγ secretion (FIG. 18). These combinations showed increased IL-4 production (FIG. 19), without statistically significant, indicating a trend towards a Th1 biased.

While the preferred embodiments of the present disclosure have been described above, it will be recognized and understood that various changes and modifications can be made, and the appended claims are intended to cover all such changes and modifications which may fall within the spirit and scope of the present disclosure.

What is claimed is:

1. An adjuvant composition, comprising:
(i) a saponin conjugate represented by formula (I) or a pharmaceutically acceptable salt or solvate thereof, wherein the formula (I) is shown as follows: 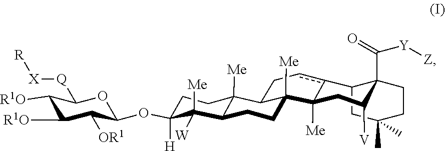
wherein: [symbol] is a single or double bond;
W is Me, —CHO, 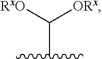
—CH2OR^{x}, or —C(O)R^{y}, wherein R^{x} is independently hydrogen or an oxygen protecting group selected from the group consisting of alkyl ethers, allyl ethers, benzyl ethers, silyl ethers, acetals, ketals, esters, carbamates, and carbonates, and R^{y} is selected from the group consisting of alkyl, allyl, benzyl, silyl, alkoxy and alkylcarboxylate;
V is H or —OH;
Y is CH₂, —O—, —S—, —NR—, or —NH—;
Q is CH₂, C═O, C═N—OH, or C═N—OMe;
X is CH₂, —O—, —NH—, —NH—(C═O)—, —(C═O)—NH—, —S—, —(C═O)—O— or —O—(C═O)—;
R is a moiety selected from the group consisting of aryl, an aryl-aliphatic group, a cyclo-aliphatic group, a heteroaryl-aliphatic group, an alkyloxy-aliphatic group, and an aryloxy-aliphatic group or a moiety selected from the group consisting of an unsubstituted 5-10-membered aryl-aliphatic group, a 5-10-membered aryl-aliphatic group substituted with at least one group selected from the group consisting of an alkyl group, an alkoxy group, an aryloxy group, a halogen, a haloalkyl group, a hydroxyl group, a saturated heterocyclic group having 1-2 heteroatoms independently selected from the group consisting of nitrogen, oxygen, and sulfur, and a combination thereof, and a 4-10-membered heteroaryl-aliphatic group having 1-4 heteroatoms independently selected from the group consisting of nitrogen, oxygen, sulfur, and a combination thereof, or having the following structures: 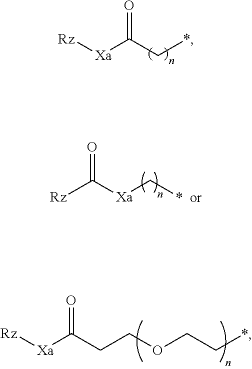
wherein Rz is alkyl, Xa is NH or S, n=1-20;
R¹ is independently hydrogen, an oxygen protecting group selected from the group consisting of alkyl ethers, allyl ethers, benzyl ethers, silyl ethers, acetals, ketals, esters, carbamates, and carbonates, or a carbohydrate having a structure of monosaccharide; and
Z is hydrogen, or a linear or branched oligosaccharide optionally substituted with at least one group selected from the group consisting of amine, amide, acyl, arylalkyl, aryl, heteroaryl, an aliphatic group, a heteroaliphatic group, a cycloaliphatic group and a heterocyclyl group; and
(ii) a TLR agonist.

2. The adjuvant composition of claim 1, wherein Z is a linear tetrasaccharide or trisaccharide, and a first sugar residue thereof is attached directly to Y.

3. The adjuvant composition of claim 1, wherein Q is C═O and X is —NH—.

4. The adjuvant composition of claim 1, wherein W is —CHO and V is —OH.

5. The adjuvant composition of claim 1, wherein R¹ is —H.

6. The adjuvant composition of claim 1, wherein R is selected from the group consisting of: 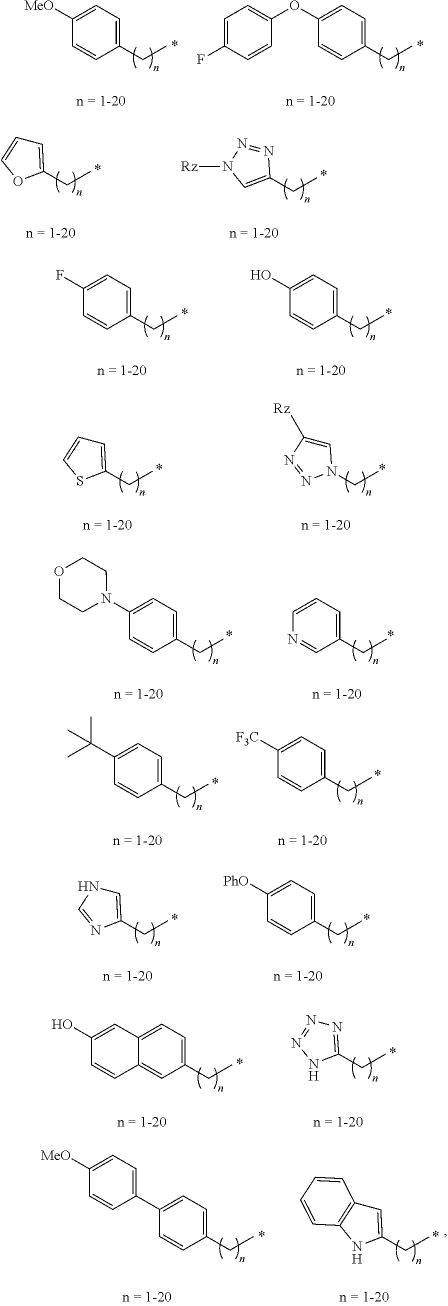
wherein Rz is alkyl.

7. The adjuvant composition of claim 1, wherein the saponin conjugate is selected from the group consisting of:
(i) a compound represented by formula I-1: 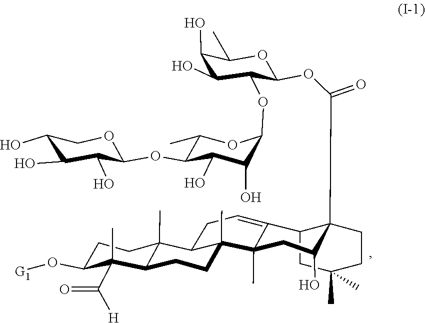
wherein G₁ is 10-((2R,3R,4S,5S,6S)-3,4,5-trihydroxy-6-((8-(4-methoxyphenyl)octyl)carbamoyl)tetrahydro-2H-pyran-2-yl), 10-((2R,3R,4S,5S,6S)-6-((8-(4-fluorophenyl)octyl)carbamoyl)-3,4,5-trihydroxytetrahydro-2H-pyran-2-yl), 10-((2R,3R,4S,5S,6S)-3,4,5-trihydroxy-6-((8-(4-phenoxyphenyl)octyl)carbamoyl)tetrahydro-2H-pyran-2-yl), 10-((2R,3R,4S,5S,6S)-6-((8-(4-(4-fluorophenoxy)phenyl)octyl)carbamoyl)-3,4,5-trihydroxytetrahydro-2H-pyran-2-yl), 10-((2R,3R,4S,5S,6S)-3,4,5-trihydroxy-6-((8-(4-morpholinophenyl)octyl)carbamoyl)tetrahydro-2H-pyran-2-yl) or 10-((2R,3R,4S,5S,6S)-6-((8-(furan-2-yl)octyl)carbamoyl)-3,4,5-trihydroxytetrahydro-2H-pyran-2-yl);
(ii) a compound represented by formula I-2: 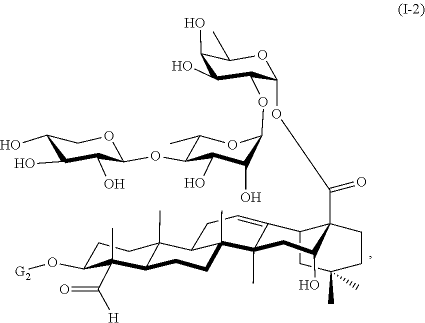
wherein G₂ is 10-((2R,3R,4S,5S,6S)-3,4,5-trihydroxy-6-((8-(4-methoxyphenyl)octyl)carbamoyl)tetrahydro-2H-pyran-2-yl), 10-((2R,3R,4S,5S,6S)-6-((8-(4-fluorophenyl)octyl)carbamoyl)-3,4,5-trihydroxytetrahydro-2H-pyran-2-yl), 10-((2R,3R,4S,5S,6S)-3,4,5-trihydroxy-6-((8-(4-phenoxyphenyl)octyl)carbamoyl)tetrahydro-2H-pyran-2-yl) or 10-((2R,3R,4S,5S,6S)-6-((8-(4-(4-fluorophenoxy)phenyl)octyl)carbamoyl)-3,4,5-trihydroxytetrahydro-2H-pyran-2-yl);
(iii) a compound represented by formula I-3: 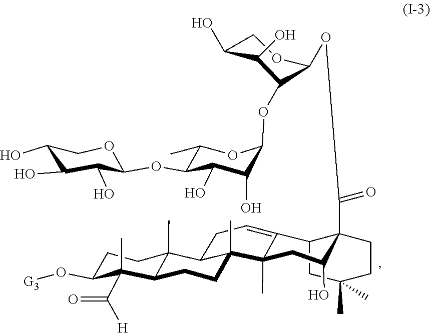
wherein G₃ is 10-((2R,3R,4S,5S,6S)-3,4,5-trihydroxy-6-((8-(4-methoxyphenyl)octyl)carbamoyl)tetrahydro-2H-pyran-2-yl), 10-((2R,3R,4S,5S,6S)-6-((8-(4-fluorophenyl)octyl)carbamoyl)-3,4,5-trihydroxytetrahydro-2H-pyran-2-yl), 10-((2R,3R,4S,5S,6S)-3,4,5-trihydroxy-6-((8-(4-phenoxyphenyl)octyl)carbamoyl)tetrahydro-2H-pyran-2-yl) or 10-((2R,3R,4S,5S,6S)-6-((8-(4-(4-fluorophenoxy)phenyl)octyl)carbamoyl)-3,4,5-trihydroxytetrahydro-2H-pyran-2-yl);
(iv) a compound represented by formula I-4: 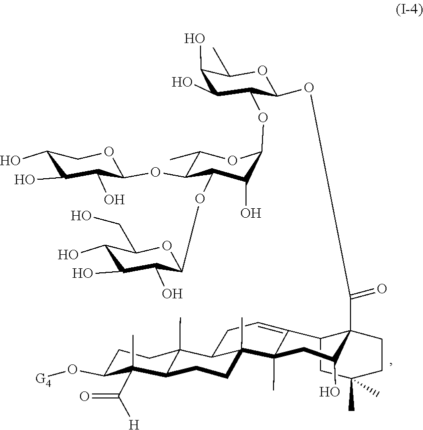
wherein G₄ is 10-((2R,3R,4S,5S,6S)-3,4,5-trihydroxy-6-((8-(4-methoxyphenyl)octyl)carbamoyl)tetrahydro-2H-pyran-2-yl), 10-((2R,3R,4S,5S,6S)-6-((8-(4-fluorophenyl)octyl)carbamoyl)-3,4,5-trihydroxytetrahydro-2H-pyran-2-yl), 10-((2R,3R,4S,5S,6S)-3,4,5-trihydroxy-6-((8-(4-phenoxyphenyl)octyl)carbamoyl)tetrahydro-2H-pyran-2-yl) or 10-((2R,3R,4S,5S,6S)-6-((8-(4-(4-fluorophenoxy)phenyl)octyl)carbamoyl)-3,4,5-trihydroxytetrahydro-2H-pyran-2-yl); and
(v) a compound represented by formula I-5: 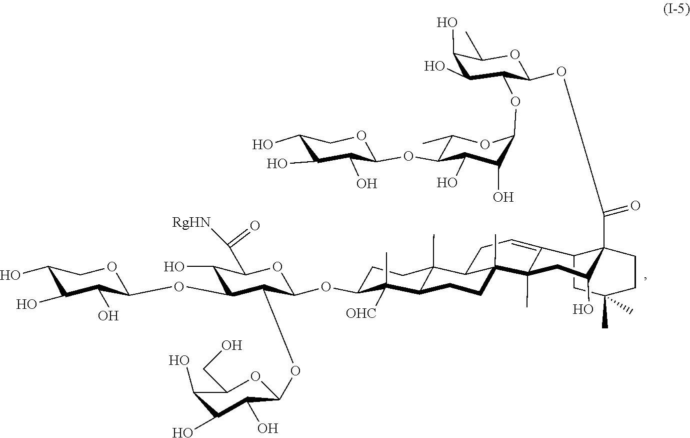
wherein Rg is 4-methoxyphenyloctyl, 4-fluorophenyloctyl, 4-phenoxyphenyloctyl or (4-fluorophenoxy)phenyl)octyl.

8. The adjuvant composition of claim 1, wherein the TLR agonist is selected from the group consisting of a TLR2 agonist, a TLR3 agonist, a TLR4 agonist, a TLR7 agonist, a TLR8 agonist, and a TLR9 agonist.

9. The adjuvant composition of claim 1, wherein the TLR agonist is a TLR4 agonist selected from the group consisting of PHAD, MPLA, 3D-PHAD, 3D-6A-PHAD, EcML, CRX-527, GSK1795091, E6020, GLA, SLA and a combination thereof.

10. The adjuvant composition of claim 1, wherein the adjuvant composition comprises liposome-forming compound or emulsion containing compound.

11. The adjuvant composition of claim 10, wherein the liposome-forming compound comprising phospholipids, cholesterol and a combination thereof.

12. The adjuvant composition of claim 10, wherein the emulsion containing compound is selected from the group consisting of polysorbate, α-tocopherol, span 85, glycerol, poloxamer 188 and carboxymethyl cellulose.

13. The adjuvant composition of claim 1, wherein the adjuvant composition is in aqueous solution, in a form of an oil in water emulsion or is encapsulated in a liposome.

14. The adjuvant composition of claim 13, wherein a particle size of the adjuvant composition is smaller than 250 nm.

15. The adjuvant composition of claim 1, further comprising squalene.

16. A pharmaceutical composition comprising an antigen and an adjuvant composition of claim 1.

17. The pharmaceutical composition of claim 16, wherein the antigen is selected form one or more of the group consisting of bacterial, viral, protozoal and tumor-related antigens.

18. A method for preventing or treating disease caused by viral infection, bacterial infection, protozoal infection or cancer, comprising a step of administering the pharmaceutical composition of claim 17 to a subject in need.
